(19) United States
(12) Patent Application Publication
(10) Pub. No.: US 2010/0143470 A1
(43) Pub. Date: Jun. 10, 2010

(54) CONTROLLED RELEASE PHARMACEUTICAL COMPOSITION CONTAINING THIAZIDES AND ANGIOTENSIN-II-RECEPTOR BLOCKERS

(75) Inventors: Sung Wuk Kim, Gyeonggi-do (KR); Sung Soo Jun, Gyeonggi-do (KR); Young Gwan Jo, Daejeon (KR); Ja Seong Koo, Daejeon (KR); Jin Wook Kim, Daejeon (KR); Jae Woon Son, Gyeonggi-do (KR)

Correspondence Address: HESLIN ROTHENBERG FARLEY & MESITI PC, 5 COLUMBIA CIRCLE, ALBANY, NY, 12203, US

(73) Assignee: HANALL PHARMACEUTICAL COMPANY, LTD, Daejeon (KR)

(21) Appl. No.: 12/447,159

(22) PCT Filed: Oct. 30, 2007
(86) PCT No.: PCT/KR2007/005403

§ 371 Date: Apr. 24, 2009

(30) Foreign Application Priority Data
Oct. 30, 2006 (KR) 10-2006-0105612

(51) Int. Cl. A61K 31/5415; A61K 47/30; A61K 47/38; A61K 47/32; A61K 9/28; A61P 9/00; A61P 13/12

(52) U.S. Cl. 424/472; 514/223.5; 514/781; 514/772.4; 514/772.6; 514/782; 424/474

(57) ABSTRACT

Disclosed herein is a pharmaceutical composition, containing a thiazide compound and an angiotensin-II-receptor blocker, and a technology for formulating the same. More particularly, disclosed is a pharmaceutical combination formulation of thiazide compound and angiotensin-II-receptor blocker, which maximizes the pharmacological and clinical antihypertensive effects and complication preventive effects of the drugs and reduces the side effects of the drugs, compared to when single-component formulations of the drugs are administered simultaneously.

### CROSS REFERENCE TO RELATED APPLICATION

This application is the National Phase filing of International Application No. PCT/KR2007/005403, filed Oct. 30, 2007, which claims priority to Korean Application No. 10-2066-0105612, filed Oct. 30, 2006. The entire content of each prior application is hereby incorporated by reference.

### TECHNICAL FIELD

The present invention relates to a pharmaceutical composition, containing a thiazide compound and an angiotensin-II-receptor blocker, and a technology for formulating the same.

More particularly, the present invention relates to a pharmaceutical combination formulation of a thiazide compound and an angiotensin-II-receptor blocker, which maximizes the pharmacological and clinical antihypertensive effects and complication preventive effects of the drugs and reduces the side effects of the drugs, compared to when single-component formulations of the drugs are co-administered simultaneously.

Particularly, the present invention relates to a technology of maximizing the antihypertensive effect of the angiotensin-II-receptor blocker by releasing the angiotension-II receptor blocker in a lag time delayed manner so as to make it possible to control blood pressure constantly from evening through bedtime to early next morning, which the technology is based on chronotherapy in which drugs are dissolved to be absorbed from the small intestine at different times in vivo.

In addition, the present invention relates to a pharmaceutical combination formulation of two drugs, which enables each of the drugs to exhibit the highest efficacy during a given time according to the biorhythmic circadian curve thereof in, such a way the antihypertensive effects and complication preventive effects of the two drugs are maintained uniformly for 24 hours.

Accordingly, the formulation of the present invention is also a novel formulation, which is based on chronotherapy and maximizes the therapeutic effects of drugs by allowing a drug combination to be administered once in the morning before 12 a.m.

Also, the present invention relates to a composition, comprising of a thiazide compound or a pharmaceutically acceptable salt thereof and an angiotensin-II-receptor blocker or a pharmaceutically acceptable salt thereof, and to a technology for formulating the composition.

Moreover, the present invention relates to a formulation, which is based on lag time delayed-release technology in addition to chronotherapy, maintains the antihypertensive effects of drugs for more than 24 hours, even when the formulation is administered once in the morning, and in addition, can improve the compliance of patients and can make it convenient for physicians to teach medication administration and for pharmacists to dispense.

### BACKGROUND ART

### Problems of Hypertension Therapy

Many antihypertensive drugs having excellent effects have been developed and used. Nevertheless, hypertension therapy is limited by the 50% rule. That is, among hypertension patients, the ratio of persons aware that they have hypertension is 50%. 50% of these patients that is only 25% receive therapy. However, only 50% of the patients who receive therapy, that is, only 12.5% of hypertension patients, receive correct therapy.

Particularly, antihypertensive therapy does not only aim to reduce the blood pressure but also to aim to prevent myocardial infarction, heart failure, stroke, premature death and the like, which are likely to arise in hypertensive patients, and to aim to prevent the condition of the diseases from being worse, thus allowing healthy longevity. To achieve such objects, antihypertensive drugs should be further improved. Also, a physician prescription should be convenient, and the compliance of patients should be easy.

Necessity of Pharmaceutical Combination Compositions

In reports of large-scale clinical trials (e.g., HOT, UKPOS, etc.), published for the past 30 years, data demonstrating that the treatment of hypertension with combination drugs from the stage of mild to severe hypertension can prevent the onset and deterioration of complications and ensure longevity have been continuously increased (Hypertension treatment guidelines, Joint National Committee on Prevention, Detection, Evaluation, and Treatment of High Blood Pressure; JNC VI & VII, WHO-ISH (1999)).

Hypertension is caused by very various factors. Various factors cause hypertension in the same patients. Thus, it is difficult to determine what results are displayed when a single antihypertensive drug is used (see Journal of human hypertension 1995: 9: S33-S36).

For these reason, the prescription of combinations of drugs has been continuously increased. Particularly, the tendency of this prescription of combinations has been clearly shown since angiotensin-II-receptor blockers (ARB: Angiotensin Receptor Blockers), such as losartan and valsartan, started to be prescribed as basic drugs.

The need to prescribe combinations of antihypertensive drugs, which have been very frequently published in the clinical and academic fields, is summarized as follows (see J. Hum. Hypertens 1995: S33-S36).

1) Hypertension is caused by various factors even in the same patients.
2) It is natural that single drug cannot treat various disease conditions.
3) The effects of a single drug are effective in only less than 50% of patients prescribed therewith.
4) The effects of combination formulations are effective in more than 80% of patients prescribed therewith.
5) Particularly, for hypertension in patients having complications such as diabetes, not only a single drug cannot attain the desired antihypertensive effect, but also it is difficult to prevent complications.
6) If a low dose of a single drug is not effective, enough to reach desirable level of blood pressure an increase in dose, in many cases, leads to an increase in side effects. However, a combination of drugs can reduce side effects.
7) When a combination of drugs having different pharmacological effects is used, it is possible to eliminate various factors and, at the same time, prevent complications and offset side effects. Thus, the American Heart Association emphasizes that it is the best treatment method to start with a combination of drugs rather than starting with a single drug.
8) Particularly, in hypertension patients having complications, the blood pressure should be lowered compared to that in patients having no complication. In this case, it is necessary to prescribe a combination of drugs. Nevertheless if a single drug is used, it can be effective in only 26% of patients. The prescription of a combination of drugs can maintain the desired blood pressure in 74% of patients, thus preventing complications from being worse (see HOT large-scale clinical trial).
9) The US FDA has recognized the necessity of combination drugs from 30 years ago on the basis of fixed-dose combination therapy. According to fixed-dose combination therapy, single drugs having different pharmacological effects should be combined with each other in the same dose as when the single drugs are prescribed alone. Such combination drugs have been approved without separate tests, as long as the effect and safety of the single drugs have already been approved, and these single drugs have been prescribed as combinations by physicians.
10) It is well known that fixed-dose combination for antihypertensive drugs have an excellent antihypertensive effect.
11) Because the doses of individual drugs are not necessarily to be increased, the incidence of the side effects of individual drugs can be significantly prevented.
12) A significant number of antihypertensive agents causes side effects in the circulatory system. Thus, in many cases, drugs having different pharmacological effects are combined with each other to offset the side effects thereof.
13) Combination drugs make the compliance of patients very easy. Thus, the amount of physician's time required to teach old people the instruction on medication can be reduced by about 50%.
14) Combination drugs can reduce risk factors for the onset of circulatory complications, and thus reduce long-term prevention cost.
15) A reduction in packaging cost for keeping each of single drugs and a reduction in time of dispensing by professional practitioners to prescribe single drugs can save the health care with big figures.

Information of Active Ingredients

The rationality for the prescription of a combination of drugs contained in the composition of the present invention and the pharmacological effects of the individual drugs are very ideal as shown in Table 1 below.

TABLE 1
Rationality for combination of drugs due to the characteristics of each
drug

| | Losartan among angiotensin-II- | Hydrochlorothiazide among |
|---|---|---|
| | receptor blockers | thiazide compounds |
| 1) Antihypertensives action | 1) by blocking the action of | 1) 12 hours after administration |
| | angiotensin-II. | shows antihypertensive action by |
| | 2) by blocking the action of | Na excretion |
| | aldosterone. | 2) 13-24 hours after administration |
| | | shows antihypertensive action by |
| | | vasodilation |
| | Because it inhibits the action of | When it is administered in the |
| | RAAS¹⁾ during sleep after | morning, it shows an |
| | midnight, it shows a strong | antihypertensive action without |
| | antihypertensive effect after | nocturnal frequent urination |
| | midnight, and thus is suitable for | |
| | non-dipper hypertensive patients.²⁾ | |
| | When the combination of two drugs are administered at different times | |
| | at about 9 am to patients in which the single drugs show insufficient | |
| | antihypertensive effects, the two drugs can show a synergistic | |
| | antihypertensive effect in non-dipper hypertensive patients during sleep. | |
| 2) Heart protection | Preventing heart failure and | Having the effect of treating |
| | myocardial infarction from being | heart failure. |
| | worse, and reducing mortality. | |
| | Synergistic action | |
| 3) Electrolyte | Inhibiting potassium loss. | Causing potassium loss. |
| | The combination of the two drugs offsets the side effects of the drugs. | |
| 4) Glucose metabolism | Reducing hyperinsulinemia. | Reducing sensitivity to diabetic |
| | | drugs |

The combination of the two drugs reduces insulin resistance in diabetic
patients.
¹⁾RAAS (Renin and Angiotensin System): one of blood pressure regulatory mechanisms in vivo
²⁾non-dipper hypertensive patients: whose blood pressure is not reduced during sleep, unlike general hypertensive patients. Old patients, diabetic patients, cardiac hypertrophy patients, etc. have a strong tendency to have non-dippers. Having a higher risk of complications such as stroke.

1) Hydrochlorothiazide, a Typical Drug Among Thiazide Diuretic Agents, and Pharmaceutical Utilization Thereof.

Hydrochlorothiazide, which is a typical thiazide diuretic agent, has a chemical name of 6-chloro-3,4-dihydro-2H-1,2,4-benzothiadiazine-7-sulfonamide-1,1-dioxide. It is administered once a day drug, and when it is orally administered for hypertension therapy, the diuretic effect is sustained for 6-12 hours for lowering blood pressure, and the blood half-time is 5.6-14.8 hours.

Hydrochlorothiazide as a hypertension therapy also shows an antihypertensive effect owing to vasodilation (vascular relaxation).

And it exerts a reduction in left atrial pressure, and a secondary antihypertensive effect through a decrease in total blood fluid by stimulating the urination (see Pharmcological Properties of Combination: Therapies for Hypertension (American Journal of Hypertension 1997; 10:13S-16S), Management of Hypertension: The Role of Combination Therapy (American Journal of Hypertension 1997; 10:262S-271S), Molecular sites for diuretic action (Bruce M. Hendry and J. Clive Elloy; TIPS-November 1988 vol. 9)].

Other thiazide diuretic agents include chlorothiazide, bendroflumethiazide and the like.

2) Angiotensin-II-Blockers and Pharmaceutical Utilization Thereof.

Angiotensin-II-receptor blockers are currently found to be drugs, which block angiotensin to bind with angiotensin receptor, one of substances causing vascular constriction, and thus exhibit an antihypertensive effect on myocardial systolic and diastolic function. A series of angiotensin-II-receptor blocker compounds, which are frequently applied in clinical trials, reach about 10 kinds, including pharmaceutically acceptable salts. Also, they are used alone or in combination with other an antihypertensive drug [see Angiotension 2 Receptor Antagonist: An Overview, Am J Health-Syst Pharm 57(13): 1231-1238, 2000].

Among such a series of angiotensin-II-receptor blockers, losartan, valsartan, irbesartan, candesartan, telmisartan, eprosartan, olmesartan and the like have been commercialized and showed rapid growth for use as hypertension treatment drugs for the past several years. Also, the effects thereof have been demonstrated through clinical trials [see Pharmacologic, Pharmacokinetic, and Therapeutic Difference Among angiotensin-II-receptor Antagonist: Pharmcotherapy 20(2): 130-139, 2000]. As demonstrated through many clinical trials, it is known that these angiotensin-II-receptor blockers show the same antihypertensive effect at the optimum dosage despite pharmacokinetic differences, such as in vivo metabolic pathways and half-life, and are also similar with respect to the effects of preventing and treating various symptoms associated with hypertension, including the effects of preventing and treating heart failure, post-myocardial infarction arrhythmia and heart failure, diabetic complications, renal failure and stoke, an antiplatelet effect, the effect of preventing arteriosclerosis, the effect of inhibiting the action of aldosterone, the effect of reducing the action of metabolic syndromes, and the effect of preventing circulatory diseases from being worse in a chain manner.

The antihypertensive and renal protective effects of the angiotensin-II-receptor blockers are described in, for example, the following publications: see J. Wagner et al.: Effects of AT1 receptor blockade on blood pressure and the renin angiotensin system in spontaneously hypertensive rats of the stroke prone strain, Clin, Exp. Hypertens., vol. 20 (1998), p. 205-221 M. Bohm et al.: Angiotensin-II-receptor blockade in TGR(mREN2)27: Effects of renin-angiotensin-system gene expression and cardiovascular functions, J. Hypertens., vol. 13(8) (1995), p. 891-899.

Other renal protective effects of angiotensin-II-receptor blockers, found in the first clinical trials, are described in the following publications: see S. Andersen et al.: Renoprotective effects of angiotensin-II-receptor blockade in type 1 diabetic patients with diabetic nephropathy, Kidney Int., vol. 57(2) (2000), p. 601-606; L. M. Ruilope: Renoprotection and renin-angiotensin system blockade in diabetes mellitus, Am. J. Hypertens., vol. 10 (12PT2) Suppl. (1997), p. 325-331.

The effects of angiotensin-II-receptor blockers on endothelial dysfunction are described in the following publications: see E. L. Schiffrin et al.: Correction of arterial structure and endothelial dysfunction in human essential hypertension by the angiotensin receptor antagonist losartan, Circulation, vol. 101(14) (2000), p. 1653-1659; R. M. Touyz et al.: Angiotensin-II-stimulates DNA and protein synthesis in vascular smooth muscle cells from human arteries: role of extracellular signal-regulated kinases, J. Hypertension., vol. 17(7) (1999), p. 907-916; E. L Schiffrin: Vascular remodelling and endothelial function in hypertensive patients: Effect of antihypertensive therapy, Scand. Cardiovasc. J., vol. 32, Suppl. 47 (1998), p. 15-21; Prasad: Acute and Chronic angiotensin-1 receptor reverses endothelial dysfunction in atherosclerosis, Circulation, vol. 101 (2000), p. 2349.

Also, Korean Patent Laid-Open Publication No. 2000-0070046 discloses that losartan among angiotensin-II-receptor blockers reduces the mortality of heart failure patients, and has the effect of preventing and reducing mortality caused by acute heart attack.

Problem of Simple Combination Therapy

1) Due to the characteristics of hypertension, the effects of antihypertensive drugs should onset in the early morning when blood pressure rises to the highest level, regardless of the type of hypertension. However, it should be recommended to administer antihypertensive drugs in the evening in order to maintain the antihypertensive action during a period between the sleeping time when the synthesis of renin causing vasoconstriction occurs and the early morning when the synthesis of angiotensin and aldosterone, acting as direct causes for blood pressure rise, reaches the highest level [see Patterns of blood pressure response: Day and night variations; American Journal of Hypertension April 2001-vol. 14, No. 4, Part 2, Preventing increase in early morning blood pressure, heart rate, and the rate-pressure, the products with controlled onset extended release verapamil at bedtime versus enalapril, losartan, and placebo on rising; American Heart Journal, October 2002]. If a simple combination of two single drugs is administered for this purpose, a sleep disorder caused by nocturnal urination, together with the deficiency of specific electrolytes such as zinc, can become severe, despite an additional antihypertensive effect is predicted by administration in combination with a diuretic agent [see Demographic, Environmental, and Genetic Predictors of Metabolic side effects of Hydrochlorothiazide treatment in Hypertensive Subjects American Journal of Hypertension 2005; 18:1077-1083].

2) If a simple combination of a diuretic agent thiazide with an angiotensin-II-receptor is administered in the morning in order to avoid a sleep disorder caused by nocturnal urination, the synthesis of vasoconstriction-causing substances during sleep cannot be strongly inhibited, and thus it is impossible to obtain the adequate antihypertensive effect of the combination drugs in the early morning when blood pressure reaches the highest level.

Examples of Prior Art

Such simple combination formulations (e.g., Hyzaar® manufactured by MSD; Diovan HCT® manufactured by Novartis, etc.), which comprise, as active ingredients, hydrochlorothiazide (thiazide diuretic agent) and an angiotensin-II-receptor blocker, are in the market. These combinations are applied to many hypertension patients, because it was clinically found that they show an additional advantage of antihypertensive effect when the two active ingredients are administered in combination. The synergistic antihypertensive action and effectiveness of the co-administration of the two active ingredients are described in detail in the following publications: see Combination Therapy in the Management of Hypertension: Focus on Angiotension Receptor blocker Combined with Diuretics, J Clin Hypertens. 2005; 7(2): 96-101, Antihypertensive Efficacy of Angiotension Receptor Blocker in Combination with Hydrochlorothiazide: A Review of the Factorial Design Study, J Clin Hypertens. 6(10): 569-577, 2004, BP circardian profile, T/P ratio and Smoothness Index After Treatment with fixed combination Losartan 100/HCTZ 25 in Essential Hypertension (American Journal of Hypertension; April 2002, vol. 15, No. 4, Part 2), Losartan prevents the Diuretics-Induced Hypokalemia and Hyperuricemia in the patients with essential Hypertension (American Journal of Hypertension; May 2005, vol. 18, No. 5, Part 2).

According to said publications, when a combination of a diuretic thiazide with an angiotensin-II-receptor blocker is administered, not only the antihypertensive effect of the angiotensin-II-receptor blocker can be obtained, but also an additional antihypertensive effect can be obtained by reducing the total plasma volume through urination. Thus, it is known that the combination can achieve a significant antihypertensive effect not only in mild hypertensive patients, but also in severe hypertensive patients who are difficult to restore to normal blood pressure through the administration of a single formulation of an angiotensin-II-receptor blocker.

Also, when hydrochlorothiazide, a thiazide diuretic agent, is administered for a long-term period of time, it can possibly show hypokalemia, because potassium is excreted through the kidneys due to side effects resulting from diuretic action, so as to cause the loss of potassium. However, the angiotensin-II-receptor block can prevent the loss of potassium by inhibiting the production of angiotensin. That is, the angiotensin-II-receptor blocker reduces side effects resulting from the long-term administration of hydrochlorothiazide acting as a thiazide diuretic agent.

The morning administration of these simple combinations can overcome a sleep disorder, which occurs due to nocturnal urination upon the evening administration of hydrochlorothiazide a thiazide diuretic agent, but the pharmacological effect of the angiotensin-II-receptor blocker cannot be maintained more strongly than when it is administered in the evening and cannot be maintained so long until the time when the risk of incidence of complications is the highest. Also, the evening administration of the combinations can maximize the pharmacological effect of the angiotensin-II-receptor blocker, but side effects, such as nocturnal urination resulting from the administration of diuretic agent hydrochlorothiazide, and a sleep disorder caused by nocturnal urination cannot be avoided.

PCT International Patent Publication No. WO 06/063737 discloses a pharmaceutical composition comprising about 80 mg telmisartan and about 25 mg hydrochlorothiazide or about 160 mg telmisartan and about 50 mg hydrochlorothiazide for the treatment of hypertension in patients with an insufficient blood pressure reduction upon treatment either with an angiotensin-II-receptor antagonist or pharmaceutical composition of low dose of hydrochlorothiazide. The disclosed composition is a simple combination of two components, in which hydrochlorothiazide and telmisartan are released simultaneously. The disclosed composition is considered to have a concept completely different from the concept of the novel composition of the present invention designed in such a way that hydrochlorothiazide is released first to exert the diuretic action during the day so as to prevent sleep disorders caused by nocturnal urination and hydrochlorothiazide penetrated into the vessel wall during the daytime could start vasodilatation from the sleeping time to exert antihypertensive action, and in such way that the angiotensin-II-receptor blocker is released at a specific time after the release of hydrochlorothiazide to exert the maximum antihypertensive effect during a period between the sleeping time when the synthesis of renin causing vasoconstriction is activated and the early morning when the synthesis of angiotensin and aldosterone, acting as direct causes for blood pressure rise. In logical or pharmacological terms, the conventional disclosed above-said compositions are considered to be an unsatisfactory combination, which cannot exhibit sufficient effects on antihypertensive and the prevention of complications. Such combinations, which are currently commercially available, are all simple combinations of two components for the optimal pharmacological effects of hydrochlorothiazide and angiotensin-II-receptor blockers could not be sufficiently exploited. Such simple combinations have been rejected due to lack of inventiveness. Korean Patent Publication No. 2000-7002144 was rejected by the KIPO, because it relates to a simple combination.

According to the present invention, a functional combination of a thiazide compound with an angiotensin-II-receptor blocker was developed for the first time in the world. The combination according to the present invention is a controlled-release pharmaceutical composition enables each of the components to exhibit the optimal pharmacological effects and can reduce the side effects of each of the components, which occur when the two components are used in combination.

### DISCLOSURE OF INVENTION

### Technical Problem

Conception of Combination Composition

Accordingly, the present inventors have developed a combination of drugs, which has an excellent antihypertensive effect compared to those of single drugs, maintains advantages, including improvement of hypokalemia reduction and an additional antihypertensive effect, through once a day administration, can overcome a sleep disorder, resulting from nocturnal urination upon simple combination therapy or the use of a simple combination of drugs, and can improve the compliance of patients through reasonable administration in the morning once a day, thereby completing the present invention.

1) It is an object of the present invention to provide a combination of a thiazide compound and an angiotensin-II-receptor blocker, which have been widely formulated and have been recognized as reasonable drugs, in which the administration of the combination drug enables the pharmacological and clinical antihypertensive effects of the drugs to be further improved and can reduce the side effects of the drugs, compared to when a thiazide single combination and an angiotensin-II-receptor blocker single combination are administered simultaneously.

2) Another object of the present invention is to activate the development of fixed-dose combinations by letting the worldwide pharmaceutical industry know how xenobiotics and chronotherapy are applied to formulation technology.

3) Still another object of the present invention is to provide a combination drug, which maintains an antihypertensive effect for more than 24 hours, even when it is administered once in the morning, and thus it can improve the compliance of patients and can make it convenient for physicians to prescribe and to teach instruction on medication administration, and increase the compliance of old patients

4) Yet another object of the present invention is to reduce the cost required for packaging single drugs and the prescription time, which increase by two times with an increase in combination prescriptions.

The present invention relates to a combination of a thiazide compound and an angiotensin-II-receptor blocker, and more particularly to a pharmaceutical composition, which is highly effective for the treatment of hypertension and comprises, as active ingredients, a thiazide diuretic agent having a long half-time in vivo and an angiotensin-II-receptor blocker showing delayed release, in such a way that the active ingredients can be administered at a specific time when the best therapeutic effect is expected, as well as a method for preparing the pharmaceutical composition.

Accordingly, the pharmaceutical composition of the present invention is a novel lag time delayed-release combination, which reduces side effects, such as hypokalemia occurring upon the administration of a thiazide diuretic agent as for hypertension therapy, and a sleep disorder caused by nocturnal urination, comprises a pharmaceutically acceptable active ingredient selected from angiotensin-II-receptor blockers, showing an excellent antihypertensive effect by vasodilation, and can release the angiotensin-II-receptor blocker in a delayed manner. The combination of the present invention shows an excellent antihypertensive effect compared when single drugs are co-administered simultaneously.

1) The thiazide compound is allowed to be absorbed from the gastrointestinal tract at a high rate immediately after administration, and the angiotensin-II-receptor blocker is allowed to be released from a given time after the absorption of the thiazide compound, in such a way that it is absorbed from the small intestine over a long period of time.

2) This combination of drugs is administered once in the morning, such that it can exhibit the effects of controlling blood pressure, inhibiting complications and reducing side effects over 24 hours.

3) The reason why the combination is administered in the morning is as follows.

The thiazide compound and the angiotensin-II-receptor blocker show the biorhythmic circadian curves during 24 hours of a day in the same manner as other drugs [see J. Clin. Hypertens 5(1): 17-23, 30, 2003].

More specifically, although the thiazide compound is sustained for 12 hours, it penetrates the vessel walls, even when it is administered only once in the morning, so that it is accumulated in the blood vessels in a given amount. This amount of thiazide compound accumulated is insufficient for sustaining the diuretic action for 24 hours, but is suitable for sustaining the vasodilatory action for 24 hours. For this reason, the 12-hr-sustained thiazide compound is currently administered only once a day.

Meanwhile, the angiotensin-II-receptor blocker shows a strong antihypertensive action between 4 p.m. and 4 a.m. for 12 hours, and thus a physiological rhythm of uniformly maintaining blood pressure during sleep at night. This is because aldostrone and angiotensin-II, which are renin systems causing an increase in blood pressure, are mainly produced at night, and the angiotensin-II-receptor blockers inhibits the production of aldosterone and the action of angiotensin-II.

Losartan, which is a typical drug among angiotensin-II-receptor blockers, enters the liver, after it is absorbed from the small intestine. A portion thereof is released into blood in the form of an active losartan molecule, which then reaches the highest blood concentration within 1 hour. However, the remaining portion is metabolized by two kinds of enzymes, cytochrome P450 2C7 and 3A4, in the liver, so as to be changed into losartan carboxylic acid (losartan's active metabolite) having higher activity, which then reaches the highest blood concentration after 3-4 hours. That is, the pharmacological action of losartan is the pharmacological action of a mixture of losartan with losartan carboxylic acid (losartan's active metabolite). About 14% of the oral dose is converted into the form of losartan carboxylic acid (active metabolite) by enzymes in the liver, and the active metabolite exhibits pharmacological activity 40 times than that of losartan. The blood elimination rate is 600 mL/min for losartan and 50 mL/min for losartan carboxylic acid (active metabolite), suggesting that the active metabolite shows a slower elimination rate, and thus plays an important role in maintaining the long-duration action time.

In the treatment of hypertensive patients, blood pressure should be maintained uniformly for 24 hours, and the excessive excitation of the heart should be inhibited uniformly for 24 hours. This object can be achieved only by the formulation technology of the present invention.

More specifically, the present invention relates to a combination of a thiazide compound and an angiotensin-II-receptor blocker, and more particularly to a pharmaceutical composition, which is very effective for the treatment of hypertension and comprises, as active ingredients, a thiazide compound having a long half-time in vivo, and an angiotensin-II-receptor blocker, which shows delayed release, in such a way that the two active ingredients can be administered simultaneously at a specific time when the best therapeutic effects can be obtained, as well as a method for preparing said pharmaceutical composition.

Accordingly, the composition of the present invention is a novel lag time delayed-release combination, which can reduce side effects, such as hypokalemia occurring upon the administration of the thiazide compound as an aid for hypertension therapy, and a sleep disorder caused by nocturnal urination, and enables the angiotensin-II-receptor blocker as an antihypertensive agent to be released in a delayed manner. The lag time delayed-release combination is a drug delivery system, which can maintain an excellent antihypertensive effect expected upon the combined administration of the two ingredients, and maintain the antihypertensive effect for more than 24 hours through the morning administration of one tablet once a day, thus improving the compliance of patients.

That is, the present inventors have found, when the novel combination for oral formulation is administered, a synergistic effect resulting from the co-administration of the thiazide compound and the angiotensin-II-receptor blocker can be maintained, and the angiotensin-II-receptor blocker can be released in a controlled manner, such that blood pressure can be controlled during a period through bedtime to next morning, and as a result, side effects, such as sleep disorders caused by nocturnal urination, and the excessive loss of electrolytes resulting from the co-administration of the active ingredients, can be reduced by controlling the administration and the in vivo metabolic rate of the combination. Also, the effect of the combination drug is maintained until the morning when blood pressure would reach higher level by stress by administering a single combination tablet in the morning once a day. So the convenience of using and the improvement of the compliance of patients may be attained. On the basis of these findings, the present invention has been completed.

### Technical Solution

The present invention relates to a combination of a thiazide compound and an angiotensin-II-receptor blocker, and more particularly to a pharmaceutical composition, which is very effective for the treatment of hypertension and comprises, as active ingredients, a thiazide diuretic agent having a long half-time in vivo, and an angiotensin-II-receptor blocker, which shows delayed release, in such a way that the ingredients can be administered simultaneously at a specific time when the best therapeutic effects are expected.

Also, the present invention encompasses a dosage form comprising an immediate release thiazide compound and a lag time delayed-release angiotensin-II-receptor blocker, which are physically separated or divided from each other, such that they may have different release rates.

Moreover, the present invention provides a novel, angiotensin-II-receptor blocker-containing granule composition capable of being compressed into a matrix for the intended lag time delayed-release of the pharmaceutically active ingredient, and provides a composition containing a thiazide compound showing immediate release.

Among the pharmaceutical compositions of the present invention, the composition containing the angiotensin-II-receptor blocker can be coated according to a conventional containing method using a release-controlling material selected from the group consisting of an enteric polymer, a water-insoluble polymer, a hydrophobic compound and a hydrophilic polymer. The coated particles or granules thus obtained and the immediate release thiazide compound-containing composition are compressed into a tablet or filled in a capsule.

As used herein, the term “controlled-release formulation” refers to a formulation in which the thiazide compound is released immediately after oral administration, such that 85% of the thiazide drug is released within 1 hour, and the angiotensin-II-receptor blocker is released in a delayed manner, such that less than 40% of the angiotensin-II-receptor blocker is released up to 4 hours. Preferably, it refers to a formulation in which less than 30% of the angiotensin-II-receptor blocker is released up to 4 hours after oral administration. This lag time delayed-release formulation can be prepared by formulating the active ingredients with a release-controlling material, a pharmaceutically acceptable diluent, a binder, a disintegrant, a lubricant, a stabilizer and the like. More preferably, the formulation is lag time delayed such that the angiotensin-II-receptor blocker is substantially released from 4 hours after the start of dissolution of the thiazide compound.

Hereinafter, the inventive pharmaceutical composition, comprising the thiazide compound and the angiotensin-II-receptor blocker, will be described in detail.

The thiazide compound is one selected from the group consisting of hydrochlorothiazide, chlorothiazide, bendroflumethiazide, and pharmaceutically acceptable salts thereof, and the angiotensin-II-receptor blocker is one selected from the group consisting of losartan, valsartan, irbesartan, candesartan, telmisartan, eprosartan, olmesartan, and pharmaceutically acceptable salts thereof, but the scope of the present invention is not limited thereto.

The dose of the thiazide compound in the composition is in the range of 5-100 mg, and the dose of the angiotensin-II-receptor blocker is in the range of 5-1200 mg. Preferably, the dose of the thiazide compound in the composition is in the range of 10-50 mg, and the dose of the angiotensin-II-receptor blocker is in the range of 8-600 mg.

The differences of the inventive functional combination drugs from a simple combination drugs, and the excellent pharmacological effects of the inventive drug combination, are summarized in Table 2 below.

TABLE 2
Comparison between functional combination and simple combination

| | | Functional combination drugs |
|---|---|---|
| | Simple combination drugs | of the present invention |
| 1) Administration time | To have been administered mainly | To be administered around 9 a.m. |
| | at 7 a.m. | after breakfast |
| 2) Dissolution and | Dissolved and absorbed | Hydrochlorothiazide: dissolved and |
| absorption of two | simultaneously in the morning | absorbed around at 9 a.m. |
| components | | Losartan: dissolved and to be started |
| | | to be absorbed around at 1 p.m. |
| 3) Prime time of stronger | Between 10 a.m. and 10 p.m. | Between 9 p.m. and 9 a.m. of the next |
| antihypertensive action | | day. |
| 4) Blood pressure control | Unsuitable | Effective for non-dippers having a |
| in non-dipper hypertensive | | high risk of incidence of |
| patients | | complications |
| 5) Time range for | (1) If it is administered | (1) If it is administered at 9 a.m. after |
| preventive effect (between | immediately after breakfast, it is | breakfast, hydrochlorothiazide will be |
| dawn and morning) having | difficult to maintain the blood | first dissolved and absorbed, so that |
| the most frequent | drug concentration in the time | an excessive amount of Na⁺ ions will |
| prevalence of the risky | when the risk of incidence of | be excreted from blood and vessel |
| complications | complications exists. | walls for 12 hours to perform |
| | (2) If it is administered around 9 | antihypertensive action. |
| | a.m., the onset of losartan will | (2) Such diuretic action is sustained |
| | start from 3 p.m., and thus, even if | up to bedtime to reduce a sleep |
| | the time when the | disorder caused by nocturnal urination |
| | antihypertensive effect is | during sleep |
| | concentrated is assumed to be 12 | (3) Hydrochlorothiazide exhibits |
| | hours, the antihypertensive effect | diuretic action during daytime, while |
| | starts to decrease after 3 a.m. | it penetrates into vessel walls and is |
| | Thus, it is unsuitable for non- | accumulated in vessel walls. Then, it |
| | dipper hypertension patients and is | exhibits vasodilatory action during |
| | unsuitable in the time range (9 | sleep to reduce blood pressure at |
| | a.m. after rising) when the risk of | night. |
| | incidence of complications is the | (4) Thus, the combination |
| | highest. | composition of the invention is |
| | | suitable for non-dipper hypertension |
| | | patients and also effective in the time |
| | | range after rising, when the risk of |
| | | incidence of complications exists, |
| | | compared to the simple combination. |

| | |
|---|---|
| 6) Interaction between the | Having no antagonism between the two components in the liver. |
| two components | |

### ADVANTAGEOUS EFFECTS

It is an object of the present invention to provide a drug delivery system, which has reduced side effects in therapeutic aspects by controlling the release of each of two active ingredients and is administered once a day in the morning to improve the compliance of patients, as well as a method for preparing the drug delivery system. The angiotensin-II-receptor blocker as a typical antihypertensive agent is lag time delayed in such a way that the release thereof is delayed for more than 3-4 hours, and preferably more than 4 hours, after the administration of the thiazide compound. When the thiazide compound having a blood half-time of 12 hours is released first, the antihypertensive action obtained by reducing the total plasma volume as well as vasodilator effect can be maintained till the next administration time. The urination which can cause a sleep disorder will occur during the day. The thiazide compound will be subjected to in vivo metabolism earlier than the angiotensin-II-receptor blocker, such that the additional loss of electrolyte, which can occur upon the combined administration of the two active ingredients, can be prevented. The angiotensin-II-receptor blocker, which is released later and absorbed over a long period of time, maintains the antihypertensive action during the time period between evening when the synthesis of vasoconstriction-causing substances occurs and morning when blood pressure reaches the highest level, such that angiotensin-II-receptor blocker shows antihypertensive action in a specific time period when the best therapeutic effect is expected. The advantages of the inventive combination drug over the simple combination drug are shown in Table 3.

TABLE 3
Superior points of inventive combination than simple combination

1) The inventive combination has an excellent effect of reducing blood
pressure.
2) The inventive combination has reduced side effects.
3) The inventive combination shows the optimal effect during the time
period when the risk of incidence of complications exists.
4) The inventive combination is most suitable for non-dipper hypertensive
patients having a high risk of incidence of complications.
5) The inventive combination realizes the right path of medication, which
reduces the time of instruction on medication.

### DESCRIPTION OF DRAWINGS

FIG. 1 is a graphic diagram showing the dissolution profiles of hydrochlorothiazide alone, losartan alone and Example 1.

FIG. 2 is a graphic diagram showing the dissolution profiles of commercially available Cozaar Plus® (simple combination of hydrochlorothiazide and losartan; Example 2).

FIG. 3 is a graphic diagram showing the dissolution profiles of Examples 5-8.

FIG. 4 is a graphic diagram showing the dissolution profiles of Examples 7 and 9-11.

FIG. 5 is a graphic diagram showing the dissolution profiles of commercially available Cozaar Plus-F® (simple combination of hydrochlorothiazide and losartan) and a formulation prepared in Example 15.

FIG. 6 is a graphic diagram showing the dissolution profiles of hydrochlorothiazide alone, losartan alone and a formulation prepared in Example 16.

FIG. 7 is a graphic diagram showing the dissolution profiles of hydrochlorothiazide alone, valsartan alone and a formulation prepared in Example 20, comprising irbesartan as an active ingredient.

FIG. 8 is a graphic diagram showing the dissolution profiles of hydrochlorothiazide alone, irbesartan alone and a formulation prepared in Example 21, comprising irbesartan as an active ingredient.

FIG. 9 is a graphic diagram showing the dissolution profiles of hydrochlorothiazide alone, losartan alone and formulations prepared in Examples 23 and 24.

FIG. 10 is a graphic diagram shows the clinical test results of Test Example 10 and indicates the comparison of systolic blood pressure between dosage methods.

FIG. 11 is a graphic diagram shows the clinical test results of Test Example 10 and indicates the comparison of diastolic blood pressure between dosage methods.

FIG. 12 is a graphic diagram shows the clinical test results of Test Example 10 and indicates the comparison of mean blood pressure between dosage methods.

### MODE FOR INVENTION

The pharmaceutical composition of the present invention can be prepared in the form of a core tablet, comprising: an inner core tablet of angiotensin-II-receptor blocker, showing release after intended delay; and an outer layer of thiazide compound, showing immediate release.

Also, the pharmaceutical composition of the present invention can be prepared in the form of a multilayer tablet, comprising: a layer of angiotensin-II-receptor blocker, showing release after intended delay; and layer of thiazide compound, showing immediate release.

This pharmaceutical composition of the present invention is suitable for the prevention and treatment of renal diseases or for the treatment of cardiovascular diseases. When it is administered between 6 a.m. and 11 a.m. once a day, it will exhibit useful effects.

The release-controlling material, which is contained in the pharmaceutical composition of the present invention, can be obtained using one selected from among an enteric polymer, a water-insoluble polymer, a hydrophobic compound and a hydrophilic polymer.

The enteric polymer may be one or a mixture of two or more selected from among polyvinyl acetate phthalate, methacrylic acid copolymers, hydroxypropylmethylcellulose phthalate, shellac, cellulose acetate phthalate, cellulose propionate phthalate, Eudragit L and Eudragit S. Preferred is hydroxypropylmethylcellulose phthalate.

The water-insoluble polymer may be one or a mixture of two or more selected from polyvinyl acetate, polymethacrylate copolymers, such as poly(ethylacrylate, methylmethacrylate) copolymers and poly(ethylacrylate, methyl methacrylate and trimethylaminoethylmethacrylate) copolymers, ethyl cellulose and cellulose acetate, which are pharmaceutically acceptable.

The hydrophobic compound may be selected from among fatty acids, fatty acid esters, fatty acid alcohols, waxes and inorganic materials. Specifically, it may be one or a mixture of two or more selected from among: fatty acids or fatty acid esters, including glyceryl palmitostearate, glyceryl stearate, glyceryl behenate, cetyl palmitate, glyceryl monooleate and stearic acid; fatty acid alcohols, including cetostearyl alcohol, cetyl alcohol and stearyl alcohol; waxes, including Carnauba wax, beeswax and microcrystalline wax; and inorganic materials, including talc, precipitated calcium carbonate, dibasic calcium phosphate, zinc oxide, titanium oxide, kaolin, bentonite, montmorillonite and veegum.

The hydrophilic polymer may be selected from among saccharides, cellulose derivatives, gums, proteins, polyvinyl derivatives, polymethacrylate copolymers, polyethylene derivatives and carboxyvinyl polymers. Specifically, it may be one or a mixture from among: saccharides, including dextrin, polydextrin, dextran, pectin and pectin derivatives, alginate, polygalacturonic acid, xylan, arabinoxylan, arabinogalactan, starch, hydroxypropyl starch, amylose and amylopectin; cellulose derivatives, including hydroxypropylmethyl cellulose, hydroxypropyl cellulose, hydroxymethyl cellulose, hydroxyethyl cellulose, methyl cellulose, carboxymethyl cellulose sodium, hydroxypropyl methylcellulose acetate succinate, and hydroxyethylmethylcellulose; gums, including guar gum, locust bean gum, tragacanth, carrageenan, gum acacia, gum arabic, gellan gum, and xanthan gum; proteins, including gelatin, casein and zein; polyvinyl derivatives, including polyvinyl alcohol, polyvinyl pyrrolidone and polyvinylacetal diethylaminoacetate; polymethacrylate copolymers, including poly(butyl methacrylate, (2-dimethylaminoethyl)methacrylate, methylmethacrylate) copolymers, poly(methacrylic acid, methylmethacrylate) copolymers, and poly(methacrylic acid, ethylacrylate) copolymers; polyethylene derivatives, including polyethylene glycol and polyethylene oxide; and carboxyvinyl polymers such as carbomer.

In addition to the above-described active ingredients and polymers, pharmaceutically acceptable dilutes, such as starch, microcrystalline cellulose, lactose, glucose, mannitol, alginate, alkaline earth metal salts, clay, polyethylene glycol and dicalcium phosphate, may be used in the tablet layer, as long as they do not impair the effects of the present invention. As binders, starch, microcrystalline cellulose, highly dispersible silica, mannitol, lactose, polyethylene glycol, polyvinyl pyrrolidone, hydroxypropyl methylcellulose, hydroxypropylcellulose, natural gum, synthetic gum, Copovidone and gelatin may be used in the inventive composition. As disintegrants, starches or modified starch such as sodium starch glycolate, corn starch, potato starch pregelatinized starch, clays such as bentonite, montmorillonite or veegum, microcrystalline cellulose, low-substituted hydroxypropylcellulose, hydroxypropylcellulose, carboxymethlycelluose, alginates such as sodium alginate, cross-linked cellulose such as croscarmellose sodium, gums such as guar gum or xanthan gum, crosslinked polymers such as crospovidone, and materials such as sodium bicarbonate or citric acid, may be used in the inventive composition. As lubricants, lubricants may include talc, magnesium stearate, alkali metal stearates such as calcium or zinc stearate, lauryl sulfate, hydrogenated vegetable oil, sodium benzoate, sodium stearyl fumarate, glyceryl monostearate and polyethyleneglycol may be used in the composition of the present invention. In addition, pharmaceutically acceptable additives selected from among colorants, fragrances and the like may be used in the present invention.

The release-controlling materials are used alone or in a mixture of two or more. They are used in a ratio of 10:0.5-1:100, preferably 5:1-1:50, and more preferably 2:1-1:30, relative to the weight of the angiotensin-II-receptor blocker. At a ratio of less than 10:0.5, it is difficult to ensure a sufficient delay time, and at a ratio of more than 1:100, the release of the drug does not occur, or the delay time exceeds 12 hours.

The scope of the present invention is not limited to the use of the above-described excipients, and these excipients may be contained in suitable amounts selected by those skilled in the art.

If necessary, a film coating layer may be formed on the outer surface of the tablet.

The novel composition of the present invention comprises: a lag time delayed-release section comprising an angiotensin-II-receptor inhibitor or a pharmaceutically acceptable salt and desired excipients; and an immediate release section comprising a thiazide compound or a pharmaceutically acceptable salt thereof and desired excipients, wherein the two sections are physically separated or divided from each other, such that the two drugs may have different release rates. The inventive composition having such physical sections can be prepared in various formulations. For example, the inventive composition can be prepared in various formulations, including an uncoated tablet, a film-coated tablet, a multilayered tablet, a core tablet, a capsule and the like.

a) A particle, granule or tablet formulation obtained by mixing an angiotensin-II-receptor blocker or a pharmaceutically acceptable salt with one or more selected from among an enteric polymer, a water-insoluble polymer, a hydrophobic compound and a hydrophilic polymer, adding pharmaceutically acceptable conventional additives thereto, and subjecting the mixture to a granulating or coating process.

b) a particle or granule formulation obtained by adding a thiazide compound or a pharmaceutically acceptable salt thereof to pharmaceutically acceptable conventional additives, and subjecting the mixture to conventional processes for producing oral solid drugs, including kneading, drying and granulation, or a formulation obtained by dissolving or suspending the particle or granule formulation in a film coating agent.

In the present invention, the delayed-release formulation shown in said a) and the immediate release formulation shown in said b) are finally contained in a single formulation.

Hereinafter, each step of a method for preparing the inventive drug delivery system showing the efficient release of a thiazide compound and an angiotensin-II-receptor blocker, will be described in detail.

Step 1)

The angiotensin-II-receptor blocker is mixed with pharmaceutically acceptable conventional additives, and the mixture is granulated. The resulting granules are used in a subsequent step without any further processing. Alternatively, the granules are compressed into a tablet using a tableting machine, and the tablet is coated with a coating solution, thus obtaining a lag time delayed-release formulation.

Step 2)

The thiazide compound is mixed with pharmaceutically acceptable conventional additives, and the mixture is subjected to conventional processes for producing oral solid formulations, including kneading, drying and granulation, thus obtaining a particle or granule formulation. If necessary, the particle or granule formulation is dissolved or suspended in a film coating agent, thus obtaining a coating solution. In this way, an immediate release formulation is obtained.

Step 3)

The particles, granules or coating solution obtained in the steps 1) and 2) are mixed with pharmaceutical excipients. The mixture is subjected to a tableting, coating or filling process, thus obtaining a formulation for oral administration.

According to this method, the inventive oral formulation showing the efficient release of the thiazide compound and the angiotensin-II-receptor blocker is prepared. More specifically, the formulation for oral administration is prepared in the following manner.

A) Preparation of Tablet

The granule formulation obtained in the step 1) is used without any further processing or is coated with a release-controlling material comprising one or more selected from among an enteric polymer, a water-insoluble polymer, a hydrophobic compound and a hydrophilic polymer. The resulting granules are mixed with the granules prepared in the step 2) and compressed into a tablet with a given amount of weight. If necessary, the obtained tablet may be coated with a film in order to improve the stability or properties thereof.

B) Preparation of Press-Coated Tablet

The tablet obtained in the step 1) is used as a core and is coated with a release-controlling material comprising one or more selected from among an enteric polymer, a water-insoluble polymer, a hydrophobic compound and a hydrophilic polymer. Then, the coated core, together with the granule obtained in the step 2), is compressed into a tablet using a press tableting machine. Alternatively, the coated core is coated with the granules obtained in the step 2), thus preparing a press-coated tablet.

C) Preparation of Multilayer Tablet

The granule obtained in the step 1) is used without any further processing or is coated with a release-controlling material comprising one or more selected from among an enteric polymer, a water-insoluble polymer, a hydrophobic compound and a hydrophilic polymer. The resulting granule is mixed with the granule obtained in the step 2) and is compressed into a bilayer tablet using a multilayer tableting machine. If necessary, a release-aid layer may be added to the bilayer tablet so as prepare a multilayer tablet having three or more layers. Alternatively, the bilayer structure may be coated to prepare a coated multilayer tablet.

D) Preparation of Film-Coated Tablet

The granule obtained in the step 1) is coated with a release-controlling material comprising one or more selected from among an enteric polymer, a water-insoluble polymer, a hydrophobic compound and a hydrophilic polymer. After drying, the granule is compressed into a tablet with a given amount of weight. Then, the tablet is coated with the drug-containing coating solution obtained in the step 2, thus preparing a film-coated tablet.

F) Capsule (Granule)

The granule obtained in the step 1) is used without any further processing. Alternatively, it is coated with a release-controlling material comprising one or more selected from among an enteric polymer, a water-insoluble polymer, a hydrophobic compound and a hydrophilic polymer and is dried. The resulting granule, together with the granule obtained in the step 2), is placed in a capsule filling machine, in which the granules are filled in a capsule in amounts corresponding to the effective amounts of the drugs, thus preparing a capsule formulation.

F) Capsule (Pellet)

An angiotensin-II-receptor blocker and a release-controlling material or pharmaceutically acceptable additives are dissolved or suspended in water or organic solvent or a mixed solvent thereof. Spherical sugar granules are coated with the solution and dried. If necessary, the granules may be coated with a solution obtained by dissolving a release-controlling material comprising one or more selected from among an enteric polymer, a water-insoluble polymer, a hydrophobic compound and a hydrophilic polymer in water, organic solvent or a mixed solvent thereof, and the coated granules may be dried. The resulting granules are coated with the coating solution obtained in the step 2), and the coated pellets are filled in a capsule. Alternatively, the coated pellets may be coated with a film, and then filled in a capsule, thus preparing a capsule formulation.

Hereinafter, the devices, detailed preparation methods and the like, used in the present invention, will be described in further detail with reference to the following examples. However, the scope of the present invention is not limited to these examples.

### Examples 1 to 14

### Preparation of Dry-Coated Tablets

1) Preparation of Losartan Lag Time Delayed-Release Cores

To prepare losartan lag time delayed-release core tablets, as shown in Table 4, losartan potassium, microcrystalline cellulose, pregelatinized starch, Copovidone and Aerosil 2000 were sieved through No. 35 sieve and mixed with each other in a high-speed mixer for 5 minutes to prepare a mixture. Magnesium stearate was mixed with the mixture for 4 minutes. The resulting mixture was compressed into core tablets using a rotary tableting machine (MRC-33, Sejong Machinery Co., Korea). The core tablets thus prepared were placed in a Hi-coater (SFC-30N, Sejong Machinery Co., Korea), in which delayed-release core tablet products having the compositions and contents shown in Tables 4 were prepared.

2) Preparation of Hydrochlorothiazide Immediate Release Layer

To prepare a hydrochlorothiazide immediate release layer, as shown in Table 4 below, hydrochlorothiazide, microcrystalline cellulose, lactose, corn starch and low-substituted hydroxypropylcellulose were weighed, sieved through No. 35 sieve and mixed with each other in a double cone mixer for 5 minutes to prepare a mixture. Meanwhile, hydroxypropylcellulose was dissolved in purified water to prepare a binder solution. The mixture together with the binder solution was placed in a fluidized bed granulator or a high-speed granulator, in which it was then granulated. Preferably, the fluidized bed granulator was used. As the fluidized bed granulator, GPCG-1 (Glatt, Germany) was used. After completion of the granulation process, the granulated material was dried in a fluidized bed dryer or a hot-water drier. Preferably, the fluidized bed dryer was used. As the fluidized bed dryer, GPCG-1 (Glatt, Germany) was used. After completion of the drying process, the dried material was sieved using an oscillator equipped with No. 18 sieve. Aerosil 200 was mixed with the sieved material in a double cone mixer. Magnesium stearate was finally mixed with the mixture in the double cone mixer.

3) Tableting and Coating

A press-coating machine (RUD-1: Kilian) was used to prepare dry-coated tablets, comprising the losartan core as an inner core layer and the hydrochlorothiazide-containing composition as an outer layer. Meanwhile, hydroxypropylmethylcellulose 2910, titanium oxide and talc were dissolved and dispersed in 80% ethanol to prepare a coating solution. Said tablets were placed in a Hi-coater (SFC-30N, Sejong Machinery Co., Korea), and then coated with the coating solution, thus preparing two-phase matrix tablets.

### Examples 15 to 22

### Preparation of Multilayered Tablets

1) Preparation of Losartan Lag Time Delayed-Release Layer

To prepare losartan lag time delayed-release core tablets, in Example 15, losartan potassium, microcrystalline cellulose, pregelatinized starch, Copovidone and sodium glycolate starch were sieved through No. 35 sieve and mixed with each other in a high-speed mixer for 5 minutes to prepare a mixture. Meanwhile, hydroxypropylcellulose and hydroxypropylcellulose phthalate (HP-50) were dissolved in purified water to prepare a binder solution. The binder solution was added to the mixture, which was then kneaded, granulated and dried. The dried granules were placed in a fluidized bed coater. Meanwhile, hydroxypropylcellulose phthalate (HP-55) and polyethyleneglycol 6000 were dissolved in 220 mg of ethanol and 980 mg of methylene chloride to prepare a coating solution. Said granules were coated with the coating solution in the fluidized bed coater (GPCG-1, Glatt, Germany). After completion of the coating process, Aerosil 200 was mixed with the coated material, and then the resulting mixture was mixed with magnesium stearate for 4 minutes, thus preparing a losartan lag time delayed-release layer.

In Examples 16 to 22, delayed-release layers having the compositions and contents shown in Table 5 were prepared according to the same method as described above.

2) Preparation of Hydrochlorothiazide Immediate Release Layers

To prepare a hydrochlorothiazide immediate release layer, hydrochlorothiazide, microcrystalline cellulose, pregelatinized starch, Copovidone and Aerosil 200 were sieved through No. 35 sieve and mixed with each other in a high-speed mixer for 5 minutes to prepare a mixture. The mixture was finally mixed with magnesium stearate in a double cone mixer for 4 minutes.

3) Tableting and Coating

A multilayer tableting machine (MRC-37T, Sejong Machinery Co., Korea) was used. The hydrochlorothiazide-containing immediate release layer composition was placed in a first powder feeder, and the losartan-containing delayed-release layer composition was placed in a second powder feeder. The compositions in the feeders were compressed into tablets in conditions in which interlayer incorporation could be minimized. Meanwhile, as the amounts shown in Table 5, hydroxypropylmethylcellulose 2910, hydroxypropylcellulose, titanium oxide and talc were dissolved and dispersed in 80% ethanol to prepare a coating solution. Said tablets were coated with the coating solution in a Hi-coater (SFC-30N, Sejong Machinery Co., Korea) to form a coating layer, thus preparing multilayered controlled-release tablets.

### Example 23

### Preparation of Film-Coated Tablets

1) Preparation of Losartan Lag Time Delayed-Release Layer

As shown in Table 5 below, losartan potassium, microcrystalline cellulose, sodium glycolate starch and lactose were sieved through No. 35 sieve and mixed with each other in a high-speed mixer for 5 minutes to prepare a mixture. Meanwhile, hydroxypropylcellulose and hydroxypropylcellulose phthalate (HP-50) were dissolved in purified water to prepare a binder solution. The binder solution was added to the mixture, which was then kneaded, granulated and dried. The dried granules were placed in a fluidized bed coater. Meanwhile, hydroxypropylcellulose phthalate (HP-55) and polyethyleneglycol 6000 were dissolved in 220 mg of ethanol and 980 mg of methylene chloride to prepare a coating solution. Said granules were coated with the coating solution in the fluidized bed coater (GPCG-1, Glatt, Germany). After completion of the coating process, Aerosil 200 was mixed with the coated material, and then the resulting mixture was mixed with magnesium stearate for 4 minutes, thus preparing a losartan delayed-release layer.

2) Preparation of Hydrochlorothiazide-Containing Immediate Release Coating Solution

Hydrochlorothiazide, hydroxypropylmethylcellulose 2910, hydroxypropylcellulose, titanium oxide and talc were dissolved and dispersed in 80% ethanol, thus preparing a hydrochlorothiazide-containing coating solution.

3) Preparation of Film-Coated Tablet, Comprising Lag Time Delayed-Release Layer of Losartan and Immediate Release Layer of Hydrochlorothiazide

The losartan delayed-release granules prepared in the step 1) were compressed into a tablet in a rotary tableting machine, and the tablet was placed in a high coater (SFC-30N, Sejong Machinery Co., Korea), in which it was then coated with the hydrochlorothiazide-containing immediate coating layer prepared in the step 2), thus preparing a controlled-release formulation in the form of a film-coated tablet, comprising the losartan delayed-release layer and the hydrochlorothiazide immediate coating layer.

### Example 24

### Preparation of Capsule

1) Preparation of Losartan Lag Time Delayed-Release Layer

As shown in Table 5 below, losartan potassium, microcrystalline cellulose, sodium glycolate starch and lactose were sieved through No. 35 sieve and mixed with each other in a high-speed mixer for 5 minutes to prepare a mixture. Meanwhile, hydroxypropylcellulose and hydroxypropylcellulose phthalate (HP-50) were dissolved in purified water to prepare a binder solution. The binder solution was added to the mixture, which was then kneaded, granulated and dried. The dried granules were placed in a fluidized bed coater. Meanwhile, hydroxypropylcellulose phthalate (HP-55) and polyethyleneglycol 6000 were dissolved in 220 mg of ethanol and 980 mg of methylene chloride to prepare a coating solution. Said granules were coated with the coating solution in the fluidized bed coater (GPCG-1, Glatt, Germany). After completion of the coating process, Aerosil 200 was mixed with the coated granules, and then the resulting material was mixed with magnesium stearate for 4 minutes, thus preparing losartan lag time delayed-release granules.

2) Preparation of Hydrochlorothiazide-Containing Immediate Release Layer

As shown in Table 5, hydrochlorothiazide, microcrystalline cellulose, pregelatinized starch, Copovidone and Aerosil 200 were sieved through No. 35 sieve and mixed with each other in a high-speed mixer for 5 minutes to prepare a mixture.

3) Mixing and Filling in Capsule

The compositions, obtained in the step 1 and 2), were mixed with each other in a double cone mixer. The mixture was finally mixed with magnesium stearate in the double cone mixer. The resulting mixture was placed in a powder feeder and filled in capsules using a capsule filling machine.

TABLE 4

| | | Composition ratio (mg/tablet) | | | | | | | | | | | | | |
|---|---|---|---|---|---|---|---|---|---|---|---|---|---|---|---|
| | | Examples | | | | | | | | | | | | | |
| Components | | 1 | 2 | 3 | 4 | 5 | 6 | 7 | 8 | 9 | 10 | 11 | 12 | 13 | 14 |
| Delayed- | Losartan | 50.0 | 50.0 | 50.0 | 50.0 | 50.0 | 50.0 | 50.0 | 50.0 | 50.0 | 50.0 | 50.0 | | | |
| release | potassium | | | | | | | | | | | | | | |
| layer | Candesartan | | | | | | | | | | | | 16 | | |
| | Telmisartan | | | | | | | | | | | | | 40 | |
| | Olmesartan | | | | | | | | | | | | | | 20 |
| | Microcrystalline | 14.0 | 14.0 | 14.0 | 14.0 | 14.0 | 14.0 | 14.0 | 14.0 | 14.0 | 14.0 | 14.0 | 14.0 | 14.0 | 14.0 |
| | cellulose | | | | | | | | | | | | | | |
| | Pregelatinized | 10.0 | 10.0 | 10.0 | 10.0 | 10.0 | 10.0 | 10.0 | 10.0 | 10.0 | 10.0 | 10.0 | 10.0 | 10.0 | 10.0 |
| | starch | | | | | | | | | | | | | | |
| | Copovidone | 4.5 | 4.5 | 4.5 | 4.5 | 4.5 | 4.5 | 4.5 | 4.5 | 4.5 | 4.5 | 4.5 | 4.5 | 4.5 | 4.5 |
| | Aerosil 200 | 1.0 | 1.0 | 1.0 | 1.0 | 1.0 | 1.0 | 1.0 | 1.0 | 1.0 | 1.0 | 1.0 | 1.0 | 1.0 | 1.0 |
| | Crosslinked | | | | | | | | | 4.0 | 8.0 | 12.0 | | | |
| | polyvinyl- | | | | | | | | | | | | | | |
| | pyrrolidone | | | | | | | | | | | | | | |
| | Hydroxypropyl | | | | | | | | | | | | | | |
| | cellulose | | | | | | | | | | | | | | |
| | Lactose | | | | | | | | | | | | | | |
| | Kollicoat | | | | | | | | | | | | | | |
| | SR30D | | | | | | | | | | | | | | |
| | Hydroxypropyl | 0.8 | 0.8 | | | | | | | | | | 0.5 | 0.7 | 0.5 |
| | methylcellulose | | | | | | | | | | | | | | |
| | Hydroxypropyl | | | | | | | | | | | | | | |
| | methylcellulose | | | | | | | | | | | | | | |
| | phthalate | | | | | | | | | | | | | | |
| | (HP-50) | | | | | | | | | | | | | | |
| | Hydroxypropyl | | | 7.2 | | | | | | | | | | | |
| | methylcellulose | | | | | | | | | | | | | | |
| | phthalate | | | | | | | | | | | | | | |
| | (HP-55) | | | | | | | | | | | | | | |
| | Polyethylene- | | | 0.8 | | | | | | | | | | | |
| | glycol 6000 | | | | | | | | | | | | | | |
| | Cellulose acetate | | | | 20.0 | | | | | | | | | | |
| | (32% acetyl | | | | | | | | | | | | | | |
| | group) | | | | | | | | | | | | | | |
| | Cellulose acetate | | | | 20.0 | | | | | | | | | | |
| | (39.8% acetyl | | | | | | | | | | | | | | |
| | group) | | | | | | | | | | | | | | |
| | Ethyl cellulose | | | | | 8.0 | 12.0 | 16.0 | 20.0 | 16.0 | 16.0 | 16.0 | | | |
| | Methacrylic acid | 8.0 | 8.0 | | | 8.0 | 8.0 | 8.0 | 8.0 | 8.0 | 8.0 | 8.0 | 4.7 | 7.1 | 5.1 |
| | copolymer type C | | | | | | | | | | | | | | |
| | Sodium chloride | | | | | | | | | | | | | | |
| | Magnesium | 1.5 | 1.5 | 1.5 | 1.5 | 1.5 | 1.5 | 1.5 | 1.5 | 1.5 | 1.5 | 1.5 | 1.5 | 1.5 | 1.5 |
| | stearate | | | | | | | | | | | | | | |
| Immediate | Hydrochloro- | 25.0 | 12.5 | 12.5 | 12.5 | 12.5 | 12.5 | 12.5 | 12.5 | 12.5 | 12.5 | 12.5 | 25.0 | 25.0 | 25.0 |
| release | thiazide | | | | | | | | | | | | | | |
| layer | Microcrystalline | 275 | 275 | 275 | 275 | 275 | 275 | 275 | 275 | 275 | 275 | 275 | 275 | 275 | 275 |
| | cellulose | | | | | | | | | | | | | | |
| | Pregelatinized | | | | | | | | | | | | | | |
| | starch | | | | | | | | | | | | | | |
| | Copovidone | | | | | | | | | | | | | | |
| | Lactose | 60.0 | 60.0 | 60.0 | 60.0 | 60.0 | 60.0 | 60.0 | 60.0 | 60.0 | 60.0 | 60.0 | 60.0 | 60.0 | 60.0 |
| | Corn starch | 90.0 | 90.0 | 90.0 | 90.0 | 90.0 | 90.0 | 90.0 | 90.0 | 90.0 | 90.0 | 90.0 | 90.0 | 90.0 | 90.0 |
| | Hydroxypropyl | 12.0 | 12.0 | 12.0 | 12.0 | 12.0 | 12.0 | 12.0 | 12.0 | 12.0 | 12.0 | 12.0 | 12.0 | 12.0 | 12.0 |
| | cellulose | | | | | | | | | | | | | | |
| | Low-substituted | 25.0 | 25.0 | 25.0 | 25.0 | 25.0 | 25.0 | 25.0 | 25.0 | 25.0 | 25.0 | 25.0 | 25.0 | 25.0 | 25.0 |
| | hydroxypropyl | | | | | | | | | | | | | | |
| | cellulose | | | | | | | | | | | | | | |
| | Aerosil 200 | 4.5 | 4.5 | 4.5 | 4.5 | 4.5 | 4.5 | 4.5 | 4.5 | 4.5 | 4.5 | 4.5 | 4.5 | 4.5 | 4.5 |
| | Magnesium | 1.0 | 1.0 | 1.0 | 1.0 | 1.0 | 1.0 | 1.0 | 1.0 | 1.0 | 1.0 | 1.0 | 1.0 | 1.0 | 1.0 |
| | stearate | | | | | | | | | | | | | | |
| Coating | Hydroxypropyl | 20.0 | 20.0 | 20.0 | 20.0 | 20.0 | 20.0 | 20.0 | 20.0 | 20.0 | 20.0 | 20.0 | 18.6 | 19.6 | 18.8 |
| layer | methylcellulose | | | | | | | | | | | | | | |
| | 2910 | | | | | | | | | | | | | | |
| | Hydroxypropyl | | | | | | | | | | | | | | |
| | cellulose | | | | | | | | | | | | | | |
| | Titanium oxide | 3.0 | 3.0 | 3.0 | 3.0 | 3.0 | 3.0 | 3.0 | 3.0 | 3.0 | 3.0 | 3.0 | 2.8 | 2.9 | 2.8 |
| | Talc | 2.0 | 2.0 | 2.0 | 2.0 | 2.0 | 2.0 | 2.0 | 2.0 | 2.0 | 2.0 | 2.0 | 1.9 | 2.0 | 1.9 |

| | | | | | | | | | | | | | | |
|---|---|---|---|---|---|---|---|---|---|---|---|---|---|---|
| Total | 607.3 | 594.8 | 594.0 | 626.0 | 602.0 | 606.0 | 610.0 | 614.0 | 614.0 | 618.0 | 622.0 | 555.5 | 583.3 | 560.1 |

TABLE 5

| | | Composition ratio (mg/tablet) | | | | | | | | | |
|---|---|---|---|---|---|---|---|---|---|---|---|
| | | Examples | | | | | | | | | |
| Components | | 15 | 16 | 17 | 18 | 19 | 20 | 21 | 22 | 23 | 24 |
| Delayed- | Losartan potassium | 100.0 | 50.0 | 50.0 | 50.0 | 50.0 | | | | 50.0 | 50.0 |
| release | Valsartan | | | | | | 80.0 | | | | |
| layer | Irbesartan | | | | | | | 150.0 | | | |
| | Eprosartan | | | | | | | | 600.0 | | |
| | Microcrystalline | 150.0 | 100.0 | 25.0 | 137.0 | 123.0 | 40.0 | 75.0 | 100.0 | 100.0 | 100.0 |
| | cellulose | | | | | | | | | | |
| | Pregelatinized starch | | | | | | | | | | |
| | Copovidone | | | | | | | | | | |
| | Sodium glycolate | 8.0 | 8.0 | | | | | | 8.0 | 8.0 | 8.0 |
| | starch | | | | | | | | | | |
| | Aerosil 200 | 2.0 | 2.0 | | | | | | 2.0 | 2.0 | 2.0 |
| | Crosslinked | | | 50.0 | | | 80.0 | 150.0 | | | |
| | polyvinylpyrrolidone | | | | | | | | | | |
| | Hydroxypropyl | 8.0 | 4.0 | 5.0 | | | 8.0 | 15.0 | 4.0 | 4.0 | 4.0 |
| | cellulose | | | | | | | | | | |
| | Lactose | 30.0 | 30.0 | | | | | | 30.0 | 30.0 | 30.0 |
| | Kollicoat SR30D | | | | | 24.0 | | | | | |
| | Hydroxypropyl | | | 2.0 | 4.0 | | 3.2 | 6.0 | | | |
| | methylcellulose | | | | | | | | | | |
| | Hydroxypropyl | 10.0 | 10.0 | | | | | | 10.0 | 10.0 | 10.0 |
| | methylcellulose | | | | | | | | | | |
| | phthalate (HP-50) | | | | | | | | | | |
| | Hydroxypropyl | 67.6 | 67.6 | | 6.0 | | | | 67.6 | 67.6 | 67.6 |
| | methylcellulose | | | | | | | | | | |
| | phthalate (HP-55) | | | | | | | | | | |
| | Polyethyleneglycol | 6.8 | 6.8 | | | | | | 6.8 | 6.8 | 6.8 |
| | 6000 | | | | | | | | | | |
| | Cellulose acetate | | | 20.0 | | | 32.0 | 60.0 | | | |
| | (32% acetyl group) | | | | | | | | | | |
| | Cellulose acetate | | | 20.0 | | | 32.0 | 60.0 | | | |
| | (39.8% acetyl group) | | | | | | | | | | |
| | Ethyl cellulose | | | | | | | | | | |
| | Methacrylic acid | | | | | | | | | | |
| | copolymer type C | | | | | | | | | | |
| | Sodium chloride | | | 25.0 | | | 40.0 | 75.0 | | | |
| | Magnesium stearate | 1.0 | 1.0 | 3.0 | 3.0 | 3.0 | 4.8 | 9.0 | 1.0 | 1.0 | 1.0 |
| Immediate | Hydrochlorothiazide | 25.0 | 12.5 | 12.5 | 12.5 | 12.5 | 12.5 | 12.5 | 12.5 | 12.5 | 12.5 |
| release | Microcrystalline | 275.0 | 51.5 | 51.5 | 51.5 | 51.5 | 51.5 | 51.5 | 51.5 | | 51.5 |
| layer | cellulose | | | | | | | | | | |
| | Pregelatinized starch | | 30.0 | 30.0 | 30.0 | 30.0 | 30.0 | 30.0 | 30.0 | | 30.0 |
| | Copovidone | | 3.0 | 3.0 | 3.0 | 3.0 | 3.0 | 3.0 | 3.0 | | 3.0 |
| | Lactose | 60.0 | | | | | | | | | |
| | Corn starch | 90.0 | | | | | | | | | |
| | Hydroxypropyl | 12.0 | | | | | | | | | |
| | cellulose | | | | | | | | | | |
| | Low-substituted | 25.0 | | | | | | | | | |
| | hydroxypropyl | | | | | | | | | | |
| | cellulose | | | | | | | | | | |
| | Aerosil 200 | 4.5 | 2.0 | 2.0 | 2.0 | 2.0 | 2.0 | 2.0 | 2.0 | | 2.0 |
| | Magnesium stearate | 1.0 | 1.0 | 1.0 | 1.0 | 1.0 | 1.0 | 1.0 | 1.0 | | 1.0 |
| Coating | Hydroxypropyl | 20.0 | 13.25 | 13.24 | 13.24 | 13.24 | 14.7 | 24.5 | 32.5 | 14.0 | |
| layer | methylcellulose 2910 | | | | | | | | | | |
| | Hydroxypropyl | | 13.25 | 13.24 | 13.24 | 13.24 | 14.7 | 24.5 | 32.5 | 14.0 | |
| | cellulose | | | | | | | | | | |
| | Titanium oxide | 3.0 | 1.9 | 1.9 | 1.9 | 1.9 | 2.2 | 3.7 | 4.9 | 2.1 | |
| | Talc | 2.0 | 1.3 | 1.3 | 1.3 | 1.3 | 1.7 | 2.8 | 3.3 | 1.6 | |

| | | | | | | | | | | |
|---|---|---|---|---|---|---|---|---|---|---|
| Total | 796.9 | 409.1 | 329.68 | 329.68 | 329.68 | 453.3 | 755.5 | 1002.6 | 323.6 | 379.4 |

### Test Example 1

### Comparative Dissolution Profile Test

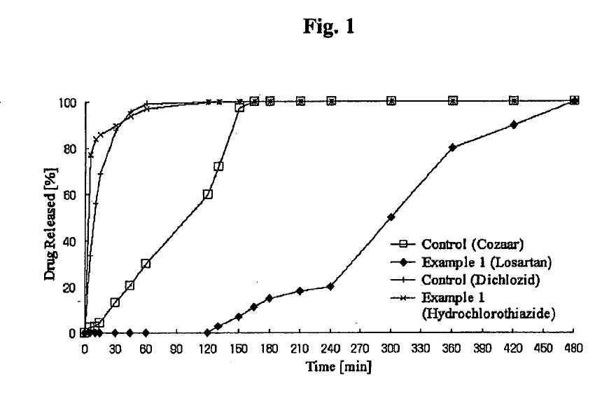

Comparative dissolution profile tests of the dry-coated tablet of losartan/hydrochlorothiazide, prepared in Example 1, and single-component control tablets (Cozaar® (MSD): single losartan tablet/Dichlozid® (Yuhan): single hydrochlorothiazide tablet), were performed. The dissolution profile test of the hydrochlorothiazide component was performed based on the United States Pharmacopoeia (USP30), and the dissolution profile test of the losartan component was performed for a total of 480 minutes, in which the dissolution medium was changed from artificial gastric juice to artificial intestinal juice starting from 120 minutes after the start of the test. The dissolution profile test of each component was performed in the following manner, and the test results are shown in FIG. 1.

As can be seen in FIG. 1, when the dissolution profile test was performed, the hydrochlorothiazide component of the dry-coated tablet of the present invention showed a dissolution profile substantially equal to that of control tablet Dichlozid®, but the losartan component showed a very slow dissolution rate compared to that of control tablet Cozaar®. In the dissolution profile test results for the losartan component, the dissolution rate of the losartan component up to 120 minutes corresponding to the artificial gastric juice zone was less than 10% in the dry-coated tablet of losartan/hydrochlorothiazide of the present invention, but was about 60% in the control formulation. The dissolution rate of the losartan component in the subsequent artificial intestinal juice zone was 100% up to a total of 150 minutes in the control tablet, but was about 20% up to a total of 240 minutes in the dry-coated tablet of losartan/hydrochlorothiazide of the present invention, suggesting that dissolution rate of the losartan component in the inventive tablet was much slower than that in the control tablet.

As described above, the early release of losartan in the dry-coated tablet of losartan/hydrochlorothiazide of the present invention is much slower than hydrochlorothiazide, unlike dissolution profiles obtained when the single losartan tablet and the single hydrochlorothiazide tablet, as the control drugs, are administered simultaneously. Accordingly, the dry-coated tablet of losartan/hydrochlorothiazide of the present invention can be administered at the point of time when the secondary antihypertensive effect of hydrochlorothiazide, and thus the inventive tablet is a pharmaceutical composition highly effective for the treatment of hypertension.

[Hydrochlorothiazide Test Method]

Dissolution profile test: performed based on the paragraph “hydrochlorothiazide tablet” in the United States Pharmacopoeia (USP30).

Test method: device 1 (paddle method), 100 rpm.

Dissolution medium: 900 ml of 0.1N hydrochloric acid.

Analysis method: UV/Vis spectrophotometry

Detection wavelength: 272 nm.

[Losartan Potassium Test Method]

Dissolution profile test: performed based on dissolution test method of general test methods in the Korean Pharmacopoeia, eighth edition.

Test method: paddle method, 50 rpm.

Dissolution media: 750 ml of 0.01M hydrochloric acid solution (artificial gastric juice); 1000 ml of pH 6.8 phosphate buffer solution (artificial intestinal juice).

Analysis method: UV/Vis spectrophotometry (detection wavelength=maximum wavelength 230 nm).

### Test Example 2

### Comparative Dissolution Profile Test

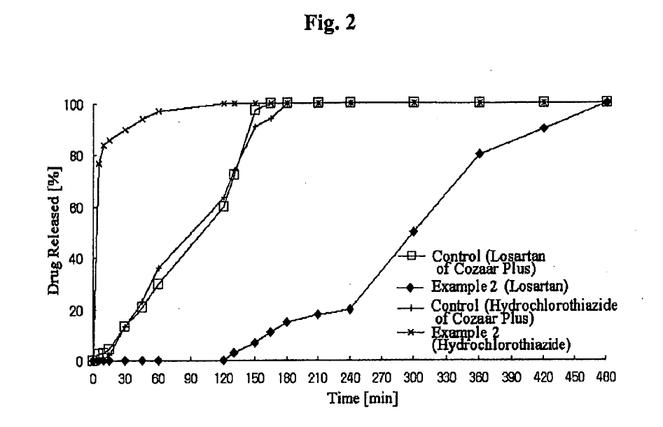

Comparative dissolution profile tests of the dry-coated tablet of losartan/hydrochlorothiazide, prepared in Example 2, and a control tablet combination (Cozaar Plus® (MSD): losartan/hydrochlorothiazide combination), were performed. The dissolution profile test of each component was performed in the same manner as in Test Example 1, and the test results are shown in FIG. 2.

As can be seen in FIG. 2, when the dissolution profile test was performed in the conditions of Example 1, the hydrochlorothiazide component of the dry-coated tablet of the present invention showed a dissolution rate faster than that of control tablet Cozaar Plus®. This is believed to be because Cozaar Plus® was a non-sectioned simple combination, unlike the single-component drug or the dry-coated tablet of the present invention, and thus the dissolution rate of hydrochlorothiazide of Cozaar Plus® was different from that of the single hydrochlorothiazide tablet due to the influence of losartan showing a slow dissolution rate in acids. In order for hydrochlorothiazide to exhibit the highest effect, hydrochlorothiazide should show a high dissolution rate similar to that of the single hydrochlorothiazide tablet, other than the delayed dissolution rate of the simple combination.

The losartan component of the inventive tablet showed a very slow dissolution rate compared to that of control tablet Cozaar Plus®, as in Test Example 1.

As described above, the dry-coated tablet of losartan/hydrochlorothiazide of the present invention shows a hydrochlorothiazide release rate is faster than that of Cozaar Plus®, and the early release of losartan in the dry-coated tablet of losartan/hydrochlorothiazide of the present invention is much slower than hydrochlorothiazide, unlike dissolution profiles obtained when the non-sectioned simple combination of losartan/hydrochlorothiazide is administered. Accordingly, the dry-coated tablet of losartan/hydrochlorothiazide of the present invention can be administered at the point of time when the secondary antihypertensive effect of hydrochlorothiazide, and thus the inventive tablet is a pharmaceutical composition highly effective for the treatment of hypertension.

### Test Example 3

### Comparative Dissolution Profile Test

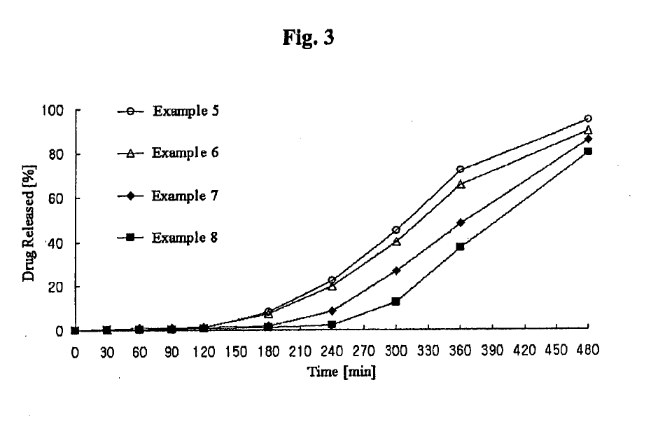

Comparative dissolution profile tests of Examples 5-8 were performed. The dissolution profile test of each component was performed in the same manner as in Test Example 1, and the test results are shown in FIG. 3.

As can be seen in FIG. 3, when the dissolution profile test was performed in the conditions of Test Example 1, the dry-coated tablet of the present invention showed a decrease in the dissolution rate of the losartan component with an increase in the amount of ethyl cellulose used. Examples 5-8 coated with ethyl cellulose showed a losartan dissolution rate of less than 20% up to a total of 240 minutes.

As described above, the early release of losartan from the inventive dry-coated tablet of losartan/hydrochlorothiazide can be delayed by the intended time by controlling the amount of ethyl cellulose used for the coating of the tablet. Accordingly, the dry-coated tablet of losartan/hydrochlorothiazide of the present invention can be administered at the point of time when the secondary antihypertensive effect of hydrochlorothiazide, and thus the inventive tablet is a pharmaceutical composition highly effective for the treatment of hypertension.

### Test Example 4

### Comparative Dissolution Profile Test

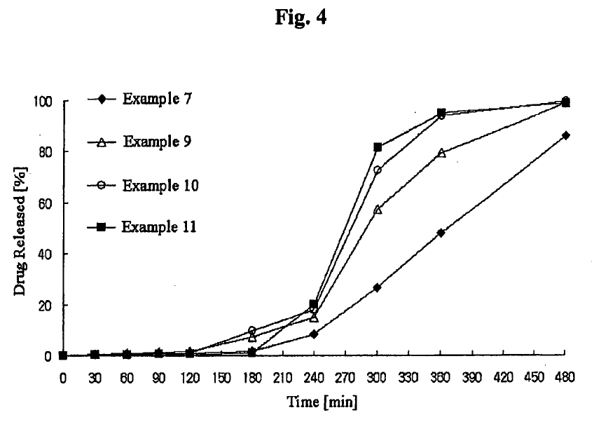

Comparative dissolution profile tests of Examples 7 and 9-11 were performed. The dissolution profile test of each component was performed in the same manner as in Test Example 1, and the test results are shown in FIG. 4.

As can be seen in FIG. 4, in the results of the dissolution profile test performed in the conditions of Test Example 1, the losartan component of the dry-coated tablet of the present invention was rapidly released after an intended delay time, when the delayed-release layer coated with ethyl cellulose contained crosslinked polyvinylpyrrolidone. The dissolution rate of the losartan component was less than 20% up to a total of 240 minutes, and the losartan component was rapidly released with an increase in the amount of crosslinked polyvinylpyrrolidone used.

As described above, the losartan component of the inventive dry-coated tablet of losartan/hydrochlorothiazide can be rapidly released after an intended delay time by controlling the amount of crosslinked polyvinylpyrrolidone used in the delayed-release layer. Accordingly, the losartan/hydrochlorothiazide dry-coated tablet of the present invention can be administered at the point of time when the secondary antihypertensive effect of hydrochlorothiazide, and thus the inventive tablet is a pharmaceutical composition highly effective for the treatment of hypertension.

### Test Example 5

### Comparative Dissolution Profile Test

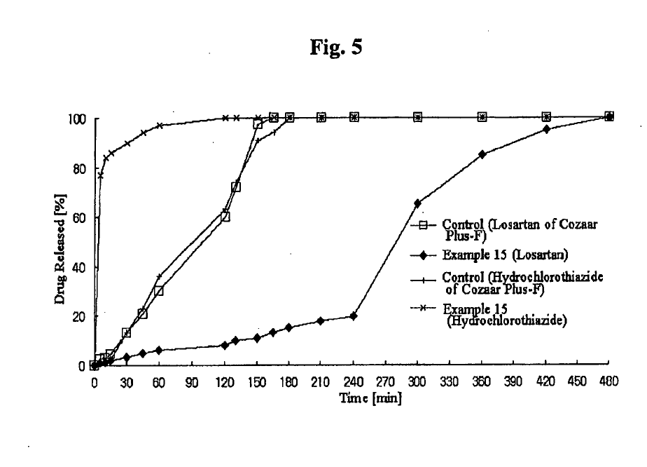

Comparative dissolution profile tests of the multilayer tablet of losartan/hydrochlorothiazide, prepared in Example 15, and a control tablet combination (Cozaar Plus-F® (MSD): losartan/hydrochlorothiazide combination), were performed. The dissolution profile test of each component was performed in the same manner as in Test Example 1, and the test results are shown in FIG. 5.

As can be seen in FIG. 5, when the dissolution profile test was performed in the conditions of Test Example 1, the hydrochlorothiazide component of the multilayer tablets of the present invention showed a dissolution rate faster than that of control tablet Cozaar Plus-F®. This is believed to be because Cozaar Plus-F® was a non-sectioned simple combination, unlike the single-component drug or the dry-coated tablet of the present invention, and thus the dissolution rate of hydrochlorothiazide of Cozaar Plus-F® was different from that of the single hydrochlorothiazide drug due to the influence of losartan showing a slow dissolution rate in acids. In order for hydrochlorothiazide to exhibit the highest effect, hydrochlorothiazide should show a high dissolution rate similar to that of the single hydrochlorothiazide drug, other than the delayed dissolution rate of the simple combination.

The losartan component of the inventive tablet showed a very slow dissolution rate compared to that of control tablet Cozaar Plus-F®, as in Test Example 1.

As described above, the losartan/hydrochlorothiazide multilayer tablet of the present invention shows a hydrochlorothiazide release rate faster than that of Cozaar Plus-F®, and the early release of losartan in the losartan/hydrochlorothiazide multilayer tablet of the present invention is much slower than hydrochlorothiazide, unlike dissolution profiles obtained when the non-sectioned simple combination of losartan/hydrochlorothiazide is administered. Accordingly, the losartan/hydrochlorothiazide multilayer tablet of the present invention can be administered at the point of time when the secondary antihypertensive effect of hydrochlorothiazide, and thus the inventive tablet is a pharmaceutical composition highly effective for the treatment of hypertension.

### Test Example 6

### Comparative Dissolution Profile Test

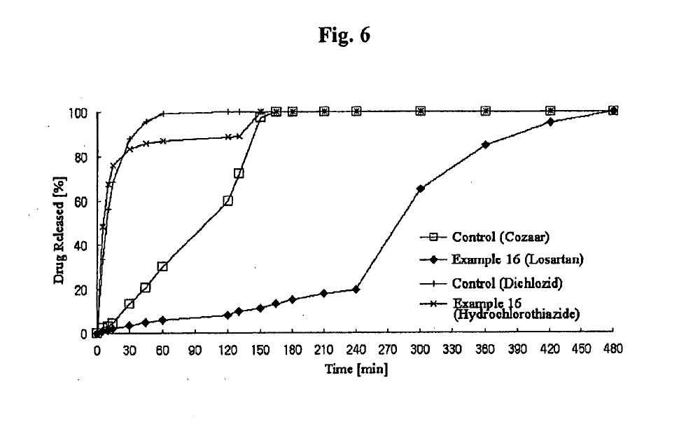

Comparative dissolution profile tests of the losartan/hydrochlorothiazide multilayer tablet, prepared in Example 16, and single-component control tablets (Cozaar® (MSD): single losartan tablet/Dichlozid® (Yuhan): single hydrochlorothiazide tablet), were performed. The dissolution profile test of each component was performed in the same manner as in Test Example 1, and the test results are shown in FIG. 6.

As can be seen in FIG. 6, when the dissolution profile test was performed in the conditions of Test Example 1, the hydrochlorothiazide component of the multilayer tablet of the present invention showed a dissolution profile substantially equal to that of control tablet Dichlozid®, but the losartan component showed a very slow dissolution rate compared to that of control tablet Cozaar®.

As described above, the early release of losartan in the losartan/hydrochlorothiazide multilayer tablet of the present invention is much slower than hydrochlorothiazide, unlike dissolution profiles obtained when the single losartan tablet and the single hydrochlorothiazide tablet, as the control drugs, are administered simultaneously. Accordingly, the losartan/hydrochlorothiazide multilayer tablet of the present invention can be administered at the point of time when the secondary antihypertensive effect of hydrochlorothiazide, and thus the inventive tablet is a pharmaceutical composition highly effective for the treatment of hypertension.

### Test Example 7

### Comparative Dissolution Profile Test

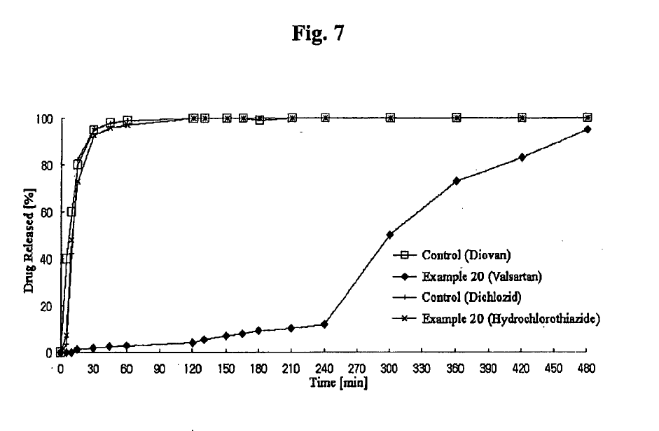

Comparative dissolution profile tests of the valsartan/hydrochlorothiazide multilayer tablet, prepared in Example 20, and single-component control tablets (Diovan® (MSD): single valsartan tablet/Dichlozid® (Yuhan): single hydrochlorothiazide tablet), were performed. The dissolution profile test of each component was performed in the same manner as in Test Example 1, and the test results are shown in FIG. 7.

As can be seen in FIG. 7, when the dissolution profile test was performed, in the condition of Test Example 1, the hydrochlorothiazide component of the multilayer tablet of the present invention showed a dissolution profile substantially equal to that of control tablet Dichlozid®, but the valsartan component showed a very slow dissolution rate compared to that of control tablet Diovan®. In the dissolution profile test results for the valsartan component, the dissolution rate of the valsartan component in the artificial intestinal juice zone was about 20% up to a total of 240 minutes in the valsartan/hydrochlorothiazide multilayer tablet of the present invention, suggesting that dissolution rate of the valsartan component in the inventive tablet was much slower than that in the control formulation.

As described above, the early release of valsartan in the valsartan/hydrochlorothiazide multilayer tablet of the present invention is much slower than hydrochlorothiazide, unlike dissolution profiles obtained when the single valsartan tablet and the single hydrochlorothiazide tablet, as the control drugs, are administered simultaneously. Accordingly, the valsartan/hydrochlorothiazide multilayer tablet of the present invention can be administered at the point of time when the secondary antihypertensive effect of hydrochlorothiazide, and thus the inventive tablet is a pharmaceutical composition highly effective for the treatment of hypertension.

### Test Example 8

### Comparative Dissolution Profile Test

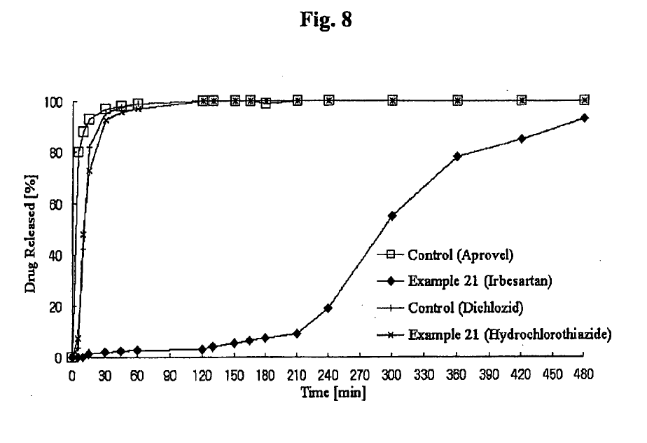

Comparative dissolution profile tests of the irbesartan/hydrochlorothiazide multilayer tablet, prepared in Example 21, and single-component control tablets (Aprovel® (MSD): single irbesartan tablet/Dichlozid® (Yuhan): single hydrochlorothiazide tablet), were performed. The dissolution profile test of each component was performed in the same manner as in Test Example 1, and the test results are shown in FIG. 8.

As can be seen in FIG. 8, when the dissolution profile test was performed, in the conditions of Test Example 1, the hydrochlorothiazide component of the multilayer tablet of the present invention showed a dissolution profile substantially equal to that of control tablet Dichlozid®, but the irbesartan component showed a very slow dissolution rate compared to that of control tablet Aprovel®. In the dissolution profile test results for the irbesartan component, the dissolution rate of the irbesartan component in the artificial intestinal juice zone was about 20% up to a total of 240 minutes in the irbesartan/hydrochlorothiazide multilayer tablet of the present invention, suggesting that dissolution rate of the irbesartan component in the inventive tablet was much slower than that in the control formulation.

As described above, the early release of irbesartan in the irbesartan/hydrochlorothiazide multilayer tablet of the present invention is much slower than hydrochlorothiazide, unlike dissolution profiles obtained when the single irbesartan tablet and the single hydrochlorothiazide tablet, as the control drugs, are administered simultaneously. Accordingly, the irbesartan/hydrochlorothiazide multilayer tablet of the present invention can be administered at the point of time when the secondary antihypertensive effect of hydrochlorothiazide, and thus the inventive tablet is a pharmaceutical composition highly effective for the treatment of hypertension.

### Test Example 9

### Comparative Dissolution Profile Test

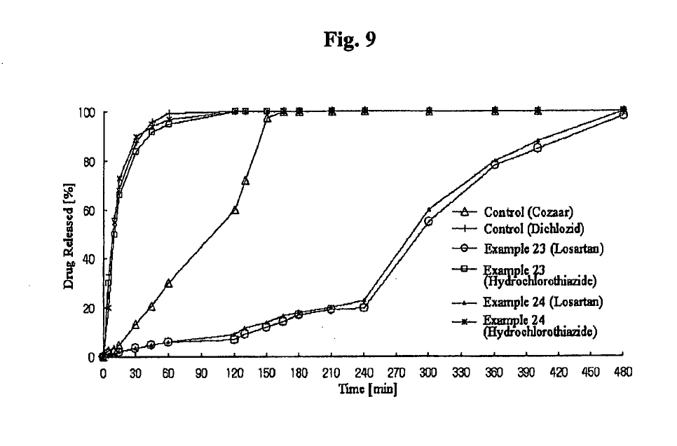

Comparative dissolution profile tests of the film-coated tablet or capsule of losartan/hydrochlorothiazide, prepared in Examples 23 and 24, and single-component control tablets (Cozaar® (MSD): single losartan tablet/Dichlozid® (Yuhan): single hydrochlorothiazide tablet), were performed. The dissolution profile test of each component was performed in the same manner as in Test Example 1, and the test results are shown in FIG. 9.

As can be seen in FIG. 9, when the dissolution profile test was performed, in the conditions of Test Example 1, the hydrochlorothiazide component of the film-coated tablet or capsule of the present invention showed a dissolution profile substantially equal to that of control tablet Dichlozid®, but the losartan component showed a very slow dissolution rate compared to that of control tablet Cozaar®. In the dissolution profile test results for the losartan component, the dissolution rate of the losartan component in the artificial intestinal juice zone was about 20% up to a total of 240 minutes in the film-coated tablet or capsule of losartan/hydrochlorothiazide of the present invention, suggesting that dissolution rate of the losartan component in the inventive tablet or capsule was much slower than that in the control tablet.

As described above, the early release of losartan in the film-coated tablet or capsule of losartan/hydrochlorothiazide of the present invention is much slower than hydrochlorothiazide, unlike dissolution profiles obtained when the single losartan tablet and the single hydrochlorothiazide tablet, as the control drugs, are administered simultaneously. Accordingly, the film-coated tablet or capsule of losartan/hydrochlorothiazide of the present invention can be administered at the point of time when the secondary antihypertensive effect of hydrochlorothiazide, and thus the inventive tablet is a pharmaceutical composition highly effective for the treatment of hypertension.

### Test Example 10

### Animal Study

In this Test Example, an animal study was performed as described in Table 6 below in order to confirm the effect of the inventive composition. Specifically, in a control group, a commercially available control drug (Cozaar Plus® (MSD): losartan/hydrochlorothiazide combination tablet) was used. In a test group, hydrochlorothiazide and losartan were administered at different times, such that the release times of the drugs were the same as in the composition provided in Example of the present invention, and thus the effects of the drugs were the same as those of the inventive composition.

Also, this animal study was designed such that the administration time showing the maximum antihypertensive effect could be confirmed.

TABLE 6

| Title | Animal study for the comparison of antihypertensive effect between the simultaneous | | |
|---|---|---|---|
| | administration of losartan and hydrochlorothiazide (HCTZ) and the administration of the | | |
| | drugs at different times in spontaneously hypertensive rats (SHR) rats. | | |
| Object | To comparatively evaluate steady-state pharmacokinetic properties, antihypertensive | | |
| | effect and safety between simultaneous administration of losartan and | | |
| | hydrochlorothiazide and the administration of the drugs at different times and to | | |
| | comparatively evaluate pharmacokinetic properties, antihypertensive effect and safety | | |
| | between administration times. | | |
| Test subjects | Twenty-five 8-week-old SHR rats grouped into five groups, each consisting of five | | |
| | animals, and four 9-week-old male Wistar Kyoto rats. | | |
| Test design | The design of this test is as follows. | | |
| | As test dugs, losartan and simvastatin were used. A total of 29 animals were grouped | | |
| | into the following six groups, each consisting of 5 animals: a saline-administered WKY | | |
| | rat group as a control group; a saline-administered SHR rat group as a screening group; a | | |
| | test group administered with losartan and hydrochlorothiazide simultaneously in the | | |
| | morning (SM group) (dark conditions); a test group administered with losartan and | | |
| | hydrochlorothiazide simultaneously in the evening (SN group) (light conditions); a test | | |
| | group administered with losartan and hydrochlorothiazide at different times in the | | |
| | morning (DM group) (dark conditions); a test group administered with losartan and | | |
| | hydrochlorothiazide at different times in the evening (DN group) (light conditions). The | | |
| | drugs were administered for 5 days once a day. | | |
| | Because this study is an animal study using rats as test models, the test was performed in | | |
| | light conditions and dark conditions. The administration time applied in the animal | | |
| | study is conversely applied to humans, because the biorhythm of rats is opposite to the | | |
| | biorhythm of humans. | | |
| Evaluation | Evaluation of effects | | |
| method | Comparison of changes in mean systolic blood pressure, mean diastolic blood pressure, | | |
| | mean blood pressure and pulse rate, measured with automatic blood pressure meter, | | |
| | between the groups administered with the drugs simultaneously in the morning and in | | |
| | evening, and the groups administered with the drugs at different times in the morning and | | |
| | in evening. | | |
| | | Administered drugs and method | |
| | | (administered at concentration | |
| | Group name | of 5 ml/kg) | Animal number |
| Test groups | Normal (WKY rats, saline) | Administered with saline hourly | 4 |
| | Vehicle (saline) | Administered with saline hourly | 5 |
| | Administered with losartan | Administered with losartan and | 5 |
| | and HCTZ simultaneously | hydrochlorothiazide | |
| | in the morning (SM group) | simultaneously at 9:30 a.m. | |
| | (dark conditions) | | |
| | Administered with losartan | Administered with losartan and | 5 |
| | and HCTZ simultaneously | hydrochlorothiazide | |
| | in the evening (SN group) | simultaneously at 7 p.m. | |
| | (light conditions) | | |
| | Administered with losartan | Administered with losartan at | 5 |
| | and HCTZ at different | 9:30 a.m.; | |
| | times in the morning (DM | Administered with | |
| | group) (dark conditions) | hydrochlorothiazide at 1:30 p.m. | |
| | Administered with losartan | Administered with losartan at 7 | 5 |
| | and HCTZ at different | p.m.; | |
| | times in the evening (DN | Administered with | |
| | group) (light conditions) | hydrochlorothiazide at 11 p.m. | |

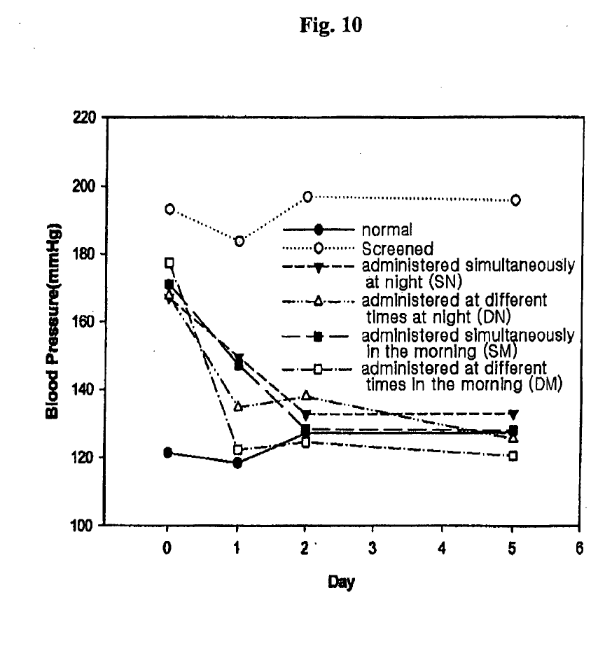

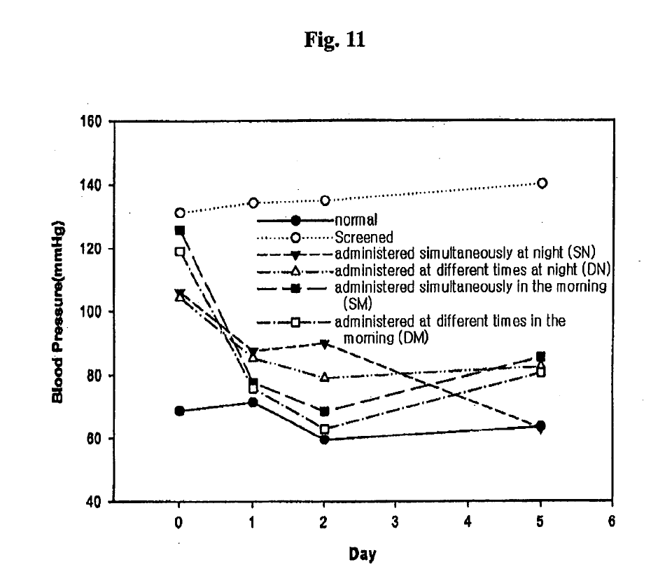

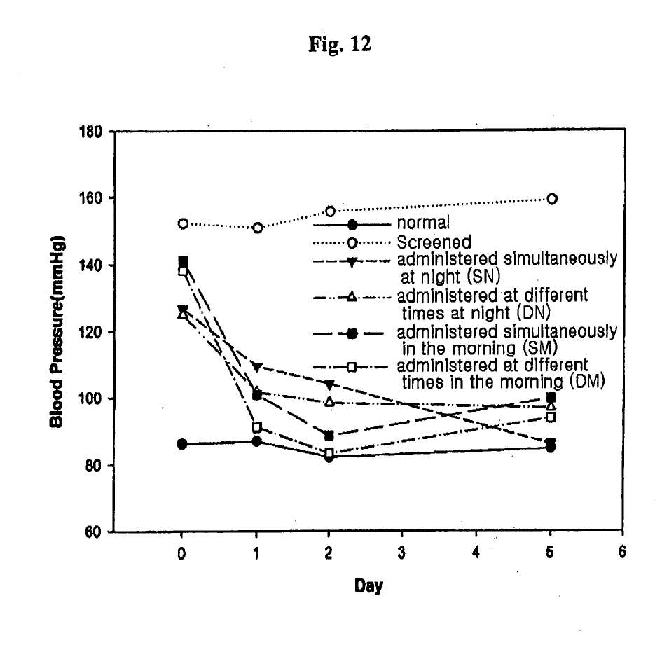

Pharmacokinetics/pharmacodynamics in the results of the clinical animal study, performed in this Test Example, is shown in Table 7 below and FIGS. 10 to 12.

TABLE 7
Results of comparative animal study between administration at different times and simultaneous administration

| | | | | | | | |
|---|---|---|---|---|---|---|---|
| 1 | Diseases | WKY rats | SHR rats | SHR rats | SHR rats | SHR rats | SHR rats |
| 2 | Groups | Normal | Triple- | Group | Group | Group | Group |
| | | group | distilled | administered | administered | administered | administered |
| | | | water | simultaneously | simultaneously | at different | at different |
| | | | | in the morning | in the evening | times in the | times in the |
| | | | | (dark | (light | morning | evening |
| | | | | conditions) | conditions) | (dark | (light |
| | | | | | | conditions) | conditions) |
| 3 | Animal number | 4 | 5 | 5 | 5 | 5 | 5 |

State of animals at 20 hours after 5-day administration

| | | | | | | | |
|---|---|---|---|---|---|---|---|
| 4 | systolic blood | 127.3 ± 4.5 | 195.8 ± 12.5 | 127.8 ± 9.2 | 132.8 ± 12.2 | 120.4 ± 7.0 | 125.4 ± 18.4 |
| | pressure | | | | | | |
| | (mmHg) | | | | | | |
| 5 | diastolic blood | 63.5 ± 12.0 | 140.2 ± 9.3 | 85.4 ± 10.2 | 63.0 ± 32.2 | 80.6 ± 15.0 | 82.4 ± 8.0 |
| | pressure | | | | | | |
| | (mmHg) | | | | | | |
| 6 | Mean blood | 84.8 ± 7.8 | 159.0 ± 6.7 | 99.6 ± 7.6 | 99.6 ± 7.6 | 93.8 ± 11.9 | 96.8 ± 2.6 |
| | pressure | | | | | | |
| | (mmHg) | | | | | | |
| 7 | Pulse rate | 466.8 ± 77.1 | 471.8 ± 44.5 | 483.8 ± 40.9 | 519.0 ± 20.8 | 484.2 ± 58.8 | 520.4 ± 57.8 |
| | (rate/min) | | | | | | |
| 8 | Clinical side | | | | Nocturnal | | Nocturnal |
| | effects | | | | urination | | urination |

This animal study was performed on rats as test models under light conditions and dark conditions. The administration time applied in the animal study is conversely applied to humans, because the biorhythm of rats is opposite to the biorhythm of humans.

1. In antihypertensive effects, systolic blood pressure and diastolic blood pressure showed low levels at day 5 compared to the screening group.

2. In the comparison of antihypertensive action between the groups administered drug simultaneously and the groups administered drugs at different times, the groups administered drugs at different times showed the lowest blood pressure level. Among the groups administered drugs at different times, the group administered in the morning (dark conditions) showed lower blood pressure level than that of the group administered in the evening (light conditions)

3. The antihypertensive effects at various times are shown in FIGS. 10 to 12. It was observed that the group administered at different times in the morning (dark conditions) showed the most excellent antihypertensive effect among the four groups.

4. In the observation of clinical side effects, the groups administered in the evening (light conditions) showed nocturnal urination, and the group administered in the morning (dark conditions) showed no urination. It can be anticipated that, in the groups administered in the morning (dark conditions), a problem of a sleep disorders caused by nocturnal urination would not occur.

Thus, it can be seen that, unlike the conventional group administered simultaneously, the composition of the present invention has the optimal antihypertensive effect during the time period from morning to midday of the day following the administration thereof, when the average blood pressure reaches the highest level.

It can be seen that, in the case of administration at different times, like the case of the inventive combination of angiotensin-II-receptor blocker/hydrochlorothiazide, the angiotensin-II-receptor blocker and hydrochlorothiazide, administered to reduce blood pressure, show an optimal antihypertensive effect compared to the case when single formulations of each of the angiotensin-II-receptor blocker and hydrochlorothiazide are simultaneously administered.

Meanwhile, Table 8 below shows the results of blood pressure level and pulse rate in the groups administered losartan and hydrochlorothiazide simultaneously and the group administered at different times in the morning (dark conditions) according to the present invention. As seen in Table 8, with respect to the antihypertensive effects of losartan and hydrochlorothiazide, the test groups administered at different times according to the present invention showed an increase of 5.8% in the effect of lowering mean systolic blood pressure, an increase of 5.6% in the effect of lowering mean diastolic blood pressure and an increase of 9.9% in the effect of lowering mean blood pressure drop, compared to those of the groups administered simultaneously, and thus the test groups showed a significant increase in the overall antihypertensive effects. Also, the test group showed an increase of 0.08% in pulse rate, but this increase was not insignificant.

Accordingly, due to the lag time on the release of losartan administered after 4 hours to reduce blood pressure as intended in the present invention, it was demonstrated that the groups administered at different times have excellent antihypertensive effects compared to the groups administered simultaneously.

TABLE 8

| | Blood pressure | Blood pressure | Blood pressure | |
|---|---|---|---|---|
| | (systolic) | (diastolic) | (mean) | Pulse rate |
| Groups | (mmHg) | (mmHg) | (mmHg) | (per min) |
| Normal group | 127.3 ± 4.5 | 63.5 ± 12.0 | 84.8 ± 7.8 | 466.8 ± 77.1 |
| Screening group | 195.8 ± 12.5 | 140.2 ± 9.3 | 159.0 ± 6.7 | 471.8 ± 44.5 |
| Administered at different times in | 120.4 ± 7.0 | 80.6 ± 15.0 | 93.8 ± 11.9 | 484.2 ± 58.8 |
| the morning (dark conditions) | | | | |
| Administered simultaneously in | 127.8 ± 9.2 | 85.4 ± 10.2 | 99.6 ± 7.6 | 483.8 ± 40.9 |
| the evening (dark conditions) | | | | |
| Difference in blood pressure drop | +5.8% | +5.6% | +9.9% | −0.08% |
| effect between group administered | | | | |
| simultaneously and group at | | | | |
| different times | | | | |

This animal study was performed on rats as test models under light/dark conditions. The administration time applied in the animal study is conversely applied to humans, because the biorhythm of rats is opposite to the biorhythm of humans.

Meanwhile, Table 9 below shows the results of blood pressure level according to the various administration times of losartan and hydrochlorothiazide. As seen in Table 9, the group administered losartan and hydrochlorothiazide in the morning (dark conditions) showed an increase of 4.0% in the effect of lowering mean systolic blood pressure, an increase of 2.2% in the effect of lowering mean diastolic blood pressure, an increase of 3.1% in the effect of lowering mean blood pressure reduction and an increase of 7.0% in pulse rate, compared to the group with losartan and hydrochlorothiazide in the evening (light conditions).

Therefore, it is proven that the group administered the combination of losartan and hydrochlorothiazide at different times in the morning (dark conditions) to reduce blood pressure as intended in the present invention has more an excellent antihypertensive effect compared to the group administered in the evening (light conditions).

TABLE 9

| | Blood pressure | Blood pressure | Blood pressure | |
|---|---|---|---|---|
| | (systolic) | (diastolic) | (mean) | Pulse rate |
| Groups | (mmHg) | (mmHg) | (mmHg) | (per min) |
| Normal group | 127.3 ± 4.5 | 63.5 ± 12.0 | 84.8 ± 7.8 | 466.8 ± 77.1 |
| Screening group | 195.8 ± 12.5 | 140.2 ± 9.3 | 159.0 ± 6.7 | 471.8 ± 44.5 |
| Administered at different times in | 120.4 ± 7.0 | 80.6 ± 15.0 | 93.8 ± 11.9 | 484.2 ± 58.8 |
| the morning (dark conditions) | | | | |
| Administered at different times in | 125.4 ± 18.4 | 82.4 ± 8.0 | 96.8 ± 2.6 | 520.4 ± 57.8 |
| the morning (light conditions) | | | | |
| Difference in blood pressure drop | +4.0% | +2.2% | +3.1% | +7.0% |
| effect between group | | | | |
| administered in evening and | | | | |
| group administered in morning | | | | |

This animal study was performed on rats as test models under light/dark conditions. The administration time applied in the animal study is conversely applied to humans, because the biorhythm of rats is opposite to the biorhythm of humans.

In conclusion, from the result of the tests above, the increased antihypertensive effect is demonstrated respectively through said clinical tests that, when the combination of angiotensin-II-receptor blocker, represented by losartan, and the thiazide compound, represented by hydrochlorothiazide, are administered at different times compared to when the drugs are administered simultaneously, when the release time of angiotensin-II-receptor is extended even at the same. Also, it was demonstrated that, when the drugs are administered in the morning (dark conditions), the antihypertensive effect is increased, compared to compared to when the drugs are administered in the evening (light conditions). Accordingly, when the drugs are administered at different times in the morning, they will show an optimal effect.

### INDUSTRIAL APPLICABILITY

The combination of drugs according to the present invention has the following advantages.

1) The inventive combination drug can perfectly exhibit physiological and clinical therapeutic effects, which are reduced when single-component formulations of a thiazide compound and an angiotensin-II-receptor blocker are co-administered simultaneously.

2) Because the inventive combination drug is administered in the morning, it can exhibit antihypertensive action and complication preventive action for 24 hours. Particularly, it can exhibit the highest antihypertensive effect in the time period when blood pressure reaches the highest level.

3) A simple method of administering the inventive combination drug in the morning will greatly contribute to the treatment of many old people patients and can make it convenient for physicians to prepare a prescription and to instruct administration of medication.

4) Because the inventive combination is a pharmaceutical combination formulation which can show the greatest effect on the prevention of three major complications, heart disease, renal disease and stroke, it will greatly contribute to the health and longevity of humans.

5) The inventive combination drug will act as the most excellent formulation against hypertension complicated with diabetes.

6) The inventive combination drug will be used as an indispensable optimal formulation in the increasing old people population.

7) Because the inventive combination drug is a combined formulation of drugs having different pharmacological properties, it can offset side effects and reduce the risk factors of incidence of circulatory complications, thus reducing long-term preventive cost.

8) A reduction in packaging cost for keeping each of single drugs and a reduction in the highly-skilled human power's time required to prescribe and to dispense single drugs can save the health care cost with big figures.

9) The inventive combination will activate the development of fixed-dose combinations by letting the worldwide pharmaceutical industry know how xenobiotics and chronotherapy are applied to formulation technology.

1. A pharmaceutical composition, for oral administration comprising:
(a) a therapeutically effective amount of a thiazide compound or a pharmaceutically acceptable salt thereof; and
(b) a therapeutically effective amount of an angiotensin-II-receptor blocker or a pharmaceutically acceptable salt thereof,
wherein, said thiazide compound or a pharmaceutically acceptable salt thereof is part of an immediate release portion and said angiotensin-II-receptor blocker or a pharmaceutically acceptable salt thereof is part of a delayed-release portion.

2. The pharmaceutical composition of claim 1, wherein said delayed-release portion comprises particles or granules comprising angiotensin-II-receptor blocker or pharmaceutically acceptable salt thereof, and a release-controlling material comprising one or more selected from an enteric polymer, a water-insoluble polymer, a hydrophobic compound and a hydrophilic polymer, and pharmaceutically acceptable excipients; and wherein said immediate-release portion comprises particles or granules comprising thiazide compound or pharmaceutically acceptable salt thereof and at least one pharmaceutically acceptable excipient.

3. The pharmaceutical composition of claim 1, wherein at least 85% of the thiazide compound or a pharmaceutically acceptable salt thereof is released within 1 hour after administration of said pharmaceutical composition to a mammal.

4. The pharmaceutical composition of claim 1, wherein the angiotensin-II-receptor blocker or a pharmaceutically acceptable salt thereof is released in a delayed manner after oral administration of the pharmaceutical composition, such that less than 40% of the angiotensin-II-receptor blocker or a pharmaceutically acceptable salt thereof is released up to 4 hours.

5. The pharmaceutical composition of claim 1, wherein the angiotensin-II-receptor blocker or a pharmaceutically acceptable salt thereof is released in a delayed manner after oral administration of the pharmaceutical composition, such that less than 30% of the angiotensin-II-receptor blocker or a pharmaceutically acceptable salt thereof is released up to 4 hours.

6. The controlled-release oral pharmaceutical composition of claim 1, wherein said thiazide compound is at least one selected from the group consisting of hydrochlorothiazide, chlorothiazide or bendroflumethiazide.

7. The controlled-release oral pharmaceutical composition of claim 6, wherein said thiazide compound is hydrochlorothiazide.

8. The controlled-release oral pharmaceutical composition of claim 1, wherein said composition comprises 5 mg to 100 mg of said thiazide compound.

9. The pharmaceutical composition of claim 1, wherein said angiotensin-II-receptor blocker is at least one selected from the group consisting of losartan, valsartan, irbesartan, candesartan, telmisartan, eprosartan or olmesartan.

10. The pharmaceutical composition of claim 9, wherein said angiotensin-II-receptor blocker is losartan.

11. The pharmaceutical composition of claim 1, wherein said composition comprises 5 mg to 1200 mg of said angiotensin-II-receptor blocker.

12. The pharmaceutical composition of claim 2, wherein said enteric polymer is at least one selected from the group consisting of polyvinyl acetate phthalate, methacrylic acid copolymers, hydroxypropylmethylcellulose phthalate, shellac, cellulose acetate phthalate, cellulose propionate phthalate, Eudragit L and Eudragit S.

13. The pharmaceutical composition of claim 2, wherein said water-insoluble polymer is at least selected from the group consisting of polyvinyl acetate, poly(ethylacrylate, methylmethacrylate) copolymers and poly(ethylacrylate, methyl methacrylate and trimethylaminoethylmethacrylate) copolymers as polymethacrylate copolymers, ethyl cellulose and cellulose acetate.

14. The pharmaceutical composition of claim 2, wherein said hydrophobic compound is at least one selected from the group consisting of fatty acids, fatty acid esters, fatty acid alcohols, waxes and inorganic materials.

15. The pharmaceutical composition of claim 14, wherein said hydrophobic compound is at least one selected form the group consisting of glyceryl palmitostearate, glyceryl stearate, glyceryl behenate, cetyl palmitate, glyceryl monooleate, stearic acid, cetostearyl alcohol, cetyl alcohol, stearyl alcohol, Carnauba wax, beeswax, microcrystalline wax, talc, precipitated calcium carbonate, dibasic calcium phosphate, zinc oxide, titanium oxide, kaolin, bentonite, montmorillonite, and veegum.

16. The pharmaceutical composition of claim 2, wherein said hydrophilic polymer is at least one selected from the group consisting of saccharides, cellulose derivatives, gums, proteins, polyvinyl derivatives, polymethacrylate copolymers, polyethylene derivatives and carboxyvinyl polymers.

17. The pharmaceutical composition of claim 16, wherein said hydrophilic polymer is at least one selected from the group consisting of dextrin, polydextrin, dextran, pectin and pectin derivatives, alginate, polygalacturonic acid, xylan, arabinoxylan, arabinogalactan, starch, hydroxypropyl starch, amylose, amylopectin hydroxypropylmethyl cellulose, hydroxypropyl cellulose, hydroxymethyl cellulose, hydroxyethyl cellulose, methyl cellulose, carboxymethyl cellulose sodium, hydroxypropyl methylcellulose acetate succinate, hydroxyethylmethylcellulose, guar gum, locust bean gum, tragacanth, carrageenan, gum acacia, gum arabic, gellan gum, xanthan gum gelatin, casein, zein polyvinyl alcohol, polyvinyl pyrrolidone, polyvinylacetal diethylaminoacetate, poly(butyl methacrylate, (2-dimethylaminoethyl)methacrylate, methylmethacrylate) copolymers, poly(methacrylic acid, methylmethacrylate) copolymers, poly(methacrylic acid, ethylacrylate) copolymers, polyethylene glycol, polyethylene oxide and carbomer.

18. The pharmaceutical composition of claim 2, wherein the weight ratio of the release-controlling material to the angiotensin-II-receptor blocker ranges from 10:0.5 to 1:100.

19. The pharmaceutical composition of claim 18, wherein the weight ratio of the release-controlling material to the angiotensin-II-receptor blocker ranges from 5:1 to 1:50.

20. The pharmaceutical composition of claim 19, wherein the weight ratio of the release-controlling material to the angiotensin-II-receptor blocker ranges from 2:1 to 1:30.

21. The pharmaceutical composition of claim 1, wherein said pharmaceutical composition is in the form of a delayed-release uncoated tablet or a film-coated tablet consisting of delayed-release granules of the angiotensin-II-receptor blocker and immediate release granules of the thiazide compound.

22. The pharmaceutical composition of claim 1, wherein said pharmaceutical composition is in the form of a press-coated tablet consisting of an immediate release-after-delayed core of the angiotensin-II-receptor blocker and an immediate release outer layer of the thiazide compound.

23. The pharmaceutical composition of claim 1, wherein said pharmaceutical composition is in the form of a multilayered tablet consisting of a delayed-release layer of the angiotensin-II-receptor blocker and an immediate release layer of the thiazide compound.

24. The pharmaceutical composition of claim 1, wherein said pharmaceutical composition is in the form of a film-coated tablet consisting of an immediate release-after-delayed uncoated layer of the angiotensin-II-receptor blocker and a film layer of the thiazide compound dissolved or suspended in a coating solution.

25. The pharmaceutical composition of claim 24, wherein said coating solution comprises a coating agent, coating aids, or a mixture thereof.

26. The pharmaceutical composition of claim 1, wherein said pharmaceutical composition is in the form of a capsule consisting of delayed-release granules of the angiotensin-II-receptor blocker and immediate release granules of the thiazide compound.

27. (canceled)

28. (canceled)

29. A method for preparing a pharmaceutical composition, which comprises the following steps:
(1) mixing or granulating an angiotensin-II-receptor blocker with pharmaceutically acceptable additives to obtain a mixture or granules, optionally compressing said granules or mixture into a tablet;
(2) mixing particles or granules of a thiazide compound with at least one pharmaceutically acceptable additive to obtain a mixture, optionally dissolving or suspending said particles or granules with a film coating agent to obtain a coating solution; and
(3) mixing the particles, granules or coating solution, obtained in the steps (1) and (2), with at least one pharmaceutically acceptable excipient, and
(4) tableting, coating or filling the mixture.

30. The pharmaceutical composition of claim 1 wherein said pharmaceutical composition is administered once a day in the morning.

31. The pharmaceutical composition according to claim 1, wherein the release of said angiotensin-II-receptor blocker begins 4 hours after the release of said thiazide compound begins.

32. The pharmaceutical composition of claim 1, wherein said angiotensin-II-receptor blocker or a therapeutically acceptable salt thereof is released 4 hours after administration of said pharmaceutical composition to a mammal.

33. The pharmaceutical composition of claim 1, wherein after administration to a mammal, said thiazide compound is substantially released before said angiotensin-II-receptor blocker is substantially released.

34. The pharmaceutical composition of claim 1, wherein after administration to a mammal, (a) said thiazide compound is substantially released within one hour, and (b) said angiotensin-II-receptor blocker is substantially released after 4 hours.

35. The pharmaceutical composition of claim 1, wherein after administration to a mammal, at least 85% of said thiazide compound is released before 40% of said angiotensin-II-receptor blocker is released.

36. The pharmaceutical composition of claim 1, wherein after administration to a mammal, at least 85% of said thiazide compound is released before 30% of said angiotensin-II-receptor blocker is released.

37. A method of treating, preventing or managing hypertension in a subject, said method comprising administering to said subject a therapeutically or prophylactically effective amount of the composition of claim 1.

38. The pharmaceutical composition of claim 1, wherein said composition is in the form of a tablet.

39. The pharmaceutical composition of claim 38, wherein said tablet is an uncoated tablet, a coated tablet, a core tablet, or a multilayered tablet.

40. The pharmaceutical composition of claim 1, wherein both the delayed-release portion and the immediate-release portion are in the form of particles or granules.

41. The pharmaceutical composition of claim 40, wherein said composition is in the form of a capsule containing said particles or granules.

42. The pharmaceutical composition of claim 1, comprising a single tablet with (a) an inner core comprising said delayed-release portion, and (b) an outer layer comprising the immediate release portion and covering the outer surface of the inner core.

43. The pharmaceutical composition according to claim 1, wherein said composition is in the form of a capsule comprising granules consisting of the delayed-release portion and granules consisting of the immediate release portion.

44. The pharmaceutical composition of claim 1, further comprising a pharmaceutically acceptable additive.

45. The pharmaceutical composition of claim 44, wherein said pharmaceutically acceptable additive is at least one selected from diluents, binders, disintegrants, lubricants, stabilizers and colorants.

46. A method of treating, preventing or managing a cardiovascular or renal disorder in a subject, comprising administering to said subject a therapeutically or prophylactically effective amount of a composition comprising: (a) a therapeutically effective amount of a thiazide compound or a pharmaceutically acceptable salt thereof; and (b) a therapeutically effective amount of an angiotensin-II-receptor blocker or a pharmaceutically acceptable salt thereof, wherein said thiazide compound or a pharmaceutically acceptable salt thereof is part of an immediate release portion and said angiotensin-II-receptor blocker or a pharmaceutically acceptable salt thereof is part of a delayed-release portion.

47. The method of claim 46, wherein said cardiovascular disorder is hypertension.

48. The method of claim 46, wherein said composition is administered once a day in the morning.

49. The method of claim 46, wherein less than 30% of said angiotensin-II-receptor blocker is released 4 hours after administration of said pharmaceutical composition to a mammal.

50. The method of claim 46, wherein less than 20% of said angiotensin-II-receptor blocker is released 4 hours after administration of said pharmaceutical composition to a mammal.

51. The method of claim 46, wherein the release of said angiotensin-II-receptor blocker begins 4 hours after administration to a mammal.
